(19) United States
(12) Patent Application Publication
(10) Pub. No.: US 2025/0122501 A1
(43) Pub. Date: Apr. 17, 2025

(54) ANTIVIRAL HAIRPIN CONSTRUCTS AND METHODS OF USE THEREOF

(71) Applicants: North Carolina State University, Raleigh, NC (US); Kansas State University Research Foundation, Manhattan, KS (US)

(72) Inventors: Anna Whitfield, Raleigh, NC (US); Dorith Rotenberg, Raleigh, NC (US); Jonathan E. Oliver, Manhattan, KS (US)

(21) Appl. No.: 18/913,587

(22) Filed: Oct. 11, 2024

Related U.S. Application Data
(60) Provisional application No. 63/590,285, filed on Oct. 13, 2023.

(51) Int. Cl. C12N 15/113; C12N 15/82

(52) U.S. Cl. CPC C12N 15/1131; C12N 15/8283; C12N 2310/14; C12N 2310/531

(57) ABSTRACT

The present disclosure provides materials and methods related to increasing viral resistance of plants. In particular, the present disclosure provides antiviral hairpin constructs having a stem region, wherein the stem region includes one or more polynucleotide sequences, wherein each of the one or more polynucleotide sequences individually have at least 70% identity or complementarity to a sequence from a target virus. Also provided are methods of increasing virus resistance or preventing viral infection in a plant comprising providing an antiviral hairpin construct or a nucleic acid encoding an antiviral hairpin construct to the plant.

### CROSS REFERENCE TO RELATED APPLICATIONS

This application claims priority to and the benefit of U.S. Provisional Patent Application No. 63/590,285 filed Oct. 13, 2023, which is incorporated herein by reference in its entirety for all purposes.

### GOVERNMENT SUPPORT

This invention was made with government support under grant numbers 58-6034-2-008 and 58-6034-7-033 awarded by the Agricultural Research Service, grant number 6034-22000-039-06S (Accession No. 0420065) awarded by the USDA/FNRI, which is funded by the USDA/ARS's Floriculture and Nursery Research Initiative, and grant numbers 2012-68004-20166 and 2016-67013-27492, awarded by the National Institute of Food and Agriculture. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 57,638 byte XML file named “NCSU_42449_202_SequenceListing” created on Oct. 8, 2024.

### FIELD

The present disclosure provides materials and methods related to increasing viral resistance of plants. In particular, the present disclosure provides antiviral hairpin constructs having a stem region, wherein the stem region includes one or more polynucleotide sequences, wherein each of the one or more polynucleotide sequences individually have at least 70% identity or complementarity to a sequence from a target virus.

### BACKGROUND

Viruses in the genus Orthotospovirus (Family Tospoviridae, Order Bunyaviriales) cause devastating diseases of important crop species throughout the world. These viruses are transmitted by multiple thrips vectors, and in the United States, cause significant viral disease epidemics on tomato, pepper, peanut, onion, soybeans, floriculture crops, and many other hosts. Several orthotospoviruses cause disease in tomato in the western hemisphere. Within the U.S., tomato spotted wilt virus (TSWV), tomato chlorotic spot virus (TCSV), groundnut ringspot virus (GRSV), Impatiens necrotic spot virus (INSV), iris yellow spot virus (IYSV), melon severe mosaic virus (MeSMV), and soybean vein necrosis virus (SVNV) are present. Within the Americas, TSWV, GRSV, INSV, TCSV, and chrysanthemum stem necrosis virus (CSNV) have been identified to cause disease in tomato. Orthotospoviruses continue to emerge and remerge as a serious threat to tomato and other vegetable production systems.

Management of orthotospovirus diseases is a persistent and dynamic challenge, but the most reliable and effective means of control is genetic resistance. In tomato, genetic resistance to orthotospoviruses is widely deployed in commercial cultivars, and relies on the Sw-5b gene, an effective source of resistance against TSWV, TCSV, and GRSV. However, in recent years, resistance-breaking (RB) orthotospovirus isolates capable of overcoming this resistance gene have been documented both in the U.S. and in other countries. Emergent RB isolates have the potential to compromise the primary means of field control of orthotospoviruses and to devastate tomato crop production.

Due to the significant losses caused by orthotospoviruses on important food crops like tomato and the limited number of genetic resistance sources available, alternative means of host resistance have been investigated, including transgenic virus resistance. Additionally, plant expression of the TSWV viral attachment protein, Gn, has been demonstrated to reduce thrips acquisition and transmission of virus. However, no transgenic virus-resistant tomato varieties are currently available commercially. Thus, there is a need for tools which enable genetic resistance to orthotospoviruses.

### SUMMARY

Embodiments of the present disclosure include RNA hairpins comprising a stem region comprising one or more polynucleotide sequences, wherein each of the one or more polynucleotide sequences individually has at least 70% (e.g., at least 75%, at least 80%, at least 85%, at least 90%, at least 95%, at least 96%, at least 97%, at least 98%, at least 99% or 100%) identity to a sequence from a target virus.

In some embodiments, the sequence from the target virus is from a conserved region of the target virus genome. In some embodiments, the conserved region includes regions of at least 25 nucleotides in which at least 85% of the nucleotide positions are conserved. In some embodiments, the conserved region includes at least two regions of at least 25 nucleotides in which at least 85% of the nucleotide positions are conserved.

In some embodiments, the sequence from the target virus is transcribed into viral mRNA. In some embodiments, the sequence from the target virus is from a protein-encoding region of the target virus. In some embodiments, the sequence from the target virus is in a domain which controls protein function.

In some embodiments, the sequence from the target virus is 100 to 200 nucleotides in length.

In some embodiments, each of the one or more polynucleotide sequences individually has at least 70% identity to a sequence from a different gene of the target virus. In some embodiments, the one or more polynucleotide sequences include in total at least one sequence with at least 70% identity to a sequence from each gene of the target virus. In some embodiments, the one or more polynucleotide sequences include in total at least one sequence with at least 70% identity to a sequence from each open reading frame of the target virus.

In some embodiments, the one or more polynucleotide sequences are selected from SEQ ID NOs: 1-10.

In some embodiments, the hairpin further comprises a loop region having a single stranded linker sequence. In some embodiments, the single stranded linker sequence is a fragment from the glucuronidase gene of E. coli.

In some embodiments, the target virus is one or more orthotospoviruses. In some embodiments, the one or more orthotospoviruses includes tomato spotted wilt virus (TSWV), tomato chlorotic spot virus (TCSV), groundnut ringspot virus (GRSV), impatiens necrotic spot virus (INSV), iris yellow spot virus (IYSV), melon severe mosaic virus (MeSMV), chrysanthemum stem necrosis virus (CSNV), soybean vein necrosis virus (SVNV), or a combination thereof. In some embodiments, the one or more orthotospovirus is a resistance-breaking orthotospovirus.

In some embodiments, the RNA hairpin is processed using endogenous cellular machinery to a cell into one or more RNA silencing compounds.

In some embodiments, RNA hairpin comprises a nucleotide sequence of any of SEQ ID NOs: 11-16.

Additional embodiments of the present disclosure include nucleic acids encoding an RNA hairpin as disclosed herein and vectors comprising a nucleic acid as disclosed herein.

Further embodiments of the present disclosure include compositions comprising an RNA hairpin as disclosed herein or a nucleic acid encoding thereof, and a carrier.

Embodiments of the present disclosure also include methods of increasing virus resistance or preventing viral infection in a plant. In some embodiments, the methods comprise providing an RNA hairpin as disclosed herein, or a nucleic acid encoding thereof, to a plant, or a plant cell, seed, fruit, plant part, or propagation material of the plant. In some embodiments, the methods further comprise providing another viral resistance treatment to the plant.

In some embodiments, the virus is one or more orthotospoviruses. In some embodiments, the one or more orthotospoviruses include tomato spotted wilt virus (TSWV), tomato chlorotic spot virus (TCSV), groundnut ringspot virus (GRSV), impatiens necrotic spot virus (INSV), iris yellow spot virus (IYSV), melon severe mosaic virus (MeSMV), chrysanthemum stem necrosis virus (CSNV), soybean vein necrosis virus (SVNV), or a combination thereof. In some embodiments, the one or more orthotospovirus is a resistance-breaking orthotospovirus.

In some embodiments, the plant is a food or ornamental crop. In some embodiments, the plant is a tomato.

Embodiments of the present disclosure further include plants, plant cells, seeds, fruit, plant parts, or propagation materials of the plant comprising an RNA hairpin as disclosed herein, or a nucleic acid encoding thereof. In some embodiments, the plant, plant cell, seed, fruit, plant part, or propagation material of the plant has increased viral resistance as compared to a plant, plant cell, seed, fruit, plant part, or propagation material of the plant not comprising the RNA hairpin or a nucleic acid encoding thereof.

Embodiments of the present disclosure further include methods of generating and/or designing an antiviral hairpin construct comprising at least one or all of: identifying conserved region of a target virus genome; selecting sequences from the conserved regions which are in protein-encoding domains; compiling selected sequences for each gene or open reading frame of the target virus into a single concatenated sequence; and generating the antiviral hairpin construct with the following regions i) a first region comprising compiled sequences, ii) a second region comprising a linker sequence configured to form a loop, and iii) a third region substantially complementary to the compiled sequences configured to form a double stranded stem with the first region.

In some embodiments, the conserved region includes regions of at least 25 nucleotides in which at least 85% of the nucleotide positions are conserved. In some embodiments, the conserved region includes at least two regions of at least 25 nucleotides in which at least 85% of the nucleotide positions are conserved.

In some embodiments, the selected sequences are in a domain which controls protein function. In some embodiments, the selected sequence for each gene or open reading frame is 100 to 200 nucleotides in length.

Other aspects and embodiments of the disclosure will be apparent in light of the following detailed description and accompanying figures.

### BRIEF DESCRIPTION OF THE DRAWINGS

FIG. 1: Bioinformatic pipeline for identification, prioritization, and selection of conserved sequence regions. The strategy targets all five genes of tomato spotted wilt virus and other related orthotospoviruses.

FIGS. 2A-2B: Hairpin construct designs. (FIG. 2A) Regions used for construct development overlaid on TSWV genomic RNAs. (FIG. 2B) Graphical depiction of the six hairpin anti-viral constructs designed including the sizes of the hairpin arms and the individual pieces used in each.

FIGS. 3A-3F: Infection incidence and plant heights of transgenic lines of tomato expressing antiviral hairpins in virus-resistance screens by mechanical, leaf-rub inoculation. Infection incidence=% plants testing positive by ELISA. (FIG. 3A) and (FIG. 3D) T2 plants challenged with a lab culture strain of a Hawaiian isolate of tomato spotted wilt virus isolate (TSWV-HI). (FIG. 3B) and (FIG. 3E) T2 plants challenged with a Florida isolate of TCSV (TCSV-FL). (FIG. 3C) and (FIG. 3F) T3 plants challenged with a California isolate of resistance-breaking tomato spotted wilt virus (RB-TSWV-CA); dpi=days post inoculation. Each bar represents the mean+standard error of the mean of data pooled from two (FIGS. 3A, 3C, 3D and 3F) or three (FIGS. 3B and 3) biological replicates of the screen (i.e., independently conducted experiments) with multiple experimental replicates (number in parentheses above bars) per transgenic line (parent) or commercial variety. Infection incidence bars headed by * indicate significant difference (P<0.05) between the transgenic test line and the transgenic negative control (shGFP=short-hairpin GFP tomato). No significant differences were determined among shGFP and the two non-transgenic controls (MM and MG=tomato cv. ‘Moneymaker’ and ‘Mountain Glory’, respectively. Plant height bars headed by a different letter indicate significant difference (P<0.05) among transgenic test lines and controls.

FIGS. 4A-4B: Evidence of hairpin transgene expression in leaves and reproductive tissues of four best-performing parental lines designed to target all viral genes of tomato spotted wilt virus and other orthotospoviruses for degradation. (FIG. 4A) Transgene expression detected in leaves, flowers, and fruits of T3 generation plants via endpoint reverse transcription-PCR with hairpin GUS-linker primers. G2.1=short-hairpin (sh) transgenic control (shGFP). No amplification was detected in the cv ‘Moneymaker’ (MM) tomato background or the “RNA” treatments which consisted of DNase-treated RNA prior to cDNA synthesis, controls that demonstrated detection of transgene RNA and not residual DNA. (FIG. 4B) Detection of transgene expression in line A3.2.3 fruit was variable across four individual fruits, but not the leaves and flowers of that plant.

FIG. 5: Thrips-mediated fruit inoculation assay to determine hairpin transgenic resistance to non-RB-TSWV and RB-TSWV. Green tomato fruits, just before ripening, were exposed to fifty viruliferous female and male (1:1) thrips (Frankliniella occidentalis) for five days. Fruit was checked for evidence of thrips feeding scars (see RB-TSWV, CA: A3.2.3 for example) and left to incubate until symptoms developed on controls (˜2 weeks) or fruit began to over-ripen (˜ 3 weeks). Black arrows indicate TSWV symptoms, either as ringspots or chlorotic clearances. Although antiviral-hairpin transgenic fruit (T3 generation plants of T2 parent lines, as shown) showed clear evidence of thrips feeding, they did not develop TSWV symptoms. Control=‘Moneymaker’, no virus-thrips control shown; shGFP=short-hairpin transgenic control; cv ‘Moneymaker’=non-transgenic control background of antiviral constructs, no Sw-5 R gene); cv. ‘Mountain Merit’=non-transgenic, Sw-5 R gene containing tomato.

FIG. 6: Table of genomic regions of high conservation between tomato spotted wilt virus and seven other orthotospovirus species. Colors represent most conserved 25 nucleotide region identified, shown below.

### DETAILED DESCRIPTION

Embodiments of the present disclosure concatenated hairpin constructs for use in developing transgenic plants with multigenic resistance to multiple orthotospovirus species and genetic variants of each species (e.g., broad spectrum resistance to orthotospoviruses). Orthotospoviruses have the capacity for rapid genetic change by genome segment reassortment and mutation. Genetic resistance is one of the most effective strategies for managing orthotospoviruses, but there are multiple examples of resistance gene breakdown. The concatenated hairpin constructs exploit the innate ability of the plant to target double-stranded RNA for degradation, a molecular process called RNA-interference (RNAi), to provide effective multigenic, broad-spectrum resistance to tomato spotted wilt virus (TSWV) and other orthotospoviruses.

The most conserved sequences for each open reading frame (ORF) of the TSWV genome were identified and comparison to other orthotospoviruses revealed sequence conservation within virus clades and some overlapped with domains with well-documented biological functions. As described herein six hairpin constructs, each of which incorporated sequences matching portions of all five ORFs. Targeting of all five viral ORFs increases the durability of resistance and combining them with other resistance genes could further extend the utility of this disease control strategy. Tomato plants expressing the hairpin transgene were challenged with TSWV by thrips and leaf-rub inoculation and four constructs provided strong protection against TSWV in foliage and fruit. The plants were challenged with tomato chlorotic spot virus and resistance-breaking TSWV (RB-TSWV) and the same constructs also provided resistance to these related viruses, indicating that the hairpin constructs disclosed herein are an effective way to protect plants from multiple orthotospoviruses and are an important strategy in the fight against RB-TSWV and emerging viruses.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” refers to plus or minus up to 10% of the value it modifies (rounded up to the nearest whole number if the value is not sub-dividable, such as a number of molecules or nucleotides). The term “about,” when modifying the quantity (e.g., mg) of a substance or composition, a parameter of a substance or composition or a parameter used in characterizing a step in a method, or the like, refers to variation in the numerical quantity that can occur. Such variation can occur through typical measuring, handling, and sampling procedures involved in the preparation, characterization and/or use of the substance or composition; through inadvertent error in these procedures; through differences in the manufacture, source, or purity of the ingredients employed to make or use the compositions or carry out the procedures; and the like.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full-length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The phrase “RNA hairpin,” as used herein, refers to an RNA-containing polynucleotide having an at least partially double stranded stem region and a single-stranded loop region. In some embodiments, the RNA hairpin comprises a sense sequence, a loop, and an antisense sequence, wherein the sense and antisense sequences are at least partially complementary and for the double stranded stem region. The sense and antisense sequences can be in different orientations with respect to one another in an RNA hairpin of the invention (L or R). An RNA hairpin can be organized in either a left-handed hairpin (e.g., 5′-antisense-loop-sense-3′) or a right-handed hairpin (e.g., 5′-sense-loop-antisense-3′). Preferably, the sense and antisense sequences are substantially complementary to each other (at least about 80% complementary). The sense sequence may also be substantially identical to a target sequence such that the antisense sequence may also be substantially complementary to a target sequence for use in RNAi (e.g., target RNA or mRNA). As described herein the sense or antisense sequence may be sequences with at least 70% identity to a sequence from a target virus. In some embodiments, the sense or antisense sequence can target multiple viral strains, although the sequence can differ from the target of a strain by one or more nucleotides (e.g., one, two, or three nucleotides). An RNA hairpin may have any length loop. The sequence of the loop can include nucleotide residues unrelated to the target. Optionally, the RNA hairpin can have an overhang region of one or more bases on either the 3′ or 5′ end of the molecule (e.g., 1 to 6 bases on the 3′ end). The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid. Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. The RNA hairpin can also comprise RNAs with stem-loop structures that contain mismatches and/or bulges.

The RNA hairpins described herein can be useful in implementing gene silencing or RNAi. Also, they may be preferred over duplexes having lengths that are similar or equivalent to the length of the stem of the hairpin in some instances, due to the fact that the s RNA hairpin described herein can be more efficient in RNA interference and less likely to induce cellular stress and/or toxicity.

The term “complementary”, as used herein, refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes.

“Perfect complementarity” or “100% complementarity”, as used herein, refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other. For example, for two 19-mers, if 17 base pairs on each strand or each region can hydrogen bond with each other, the polynucleotide strands exhibit 89.5% complementarity. Substantial complementarity refers to polynucleotide strands or regions exhibiting about 80% or greater complementarity.

The terms “RNA interference” and “RNAi” are used interchangeably herein, and refer to the process by which a molecule exerts an effect on a biological process by interacting with one or more components of the RNAi pathway including, but not limited to, Drosha, Dicer, Argonaute family proteins, etc. The process includes, but is not limited to, gene silencing by degrading mRNA; attenuating translation, interactions with tRNA, rRNA, hnRNA, cDNA and genomic DNA; and inhibiting as well as methylating DNA with ancillary proteins.

As used herein, the terms “resistance,” “resistant,” and “host plant resistance” are used interchangeably herein, and refer to the ability of a host plant, plant cell or plant part to prevent or reduce infestation and damage of a pest from the group comprising insects, nematodes, pathogens, fungi, viruses, and diseases. As such, the terms “virus resistance,” viral resistant,” and the like specifically refer to the ability of a host plant, plant cell or plant part to prevent or reduce viral infection.

A “peptide” or “polypeptide” is a linked sequence of two or more amino acids linked by peptide bonds. Peptides and polypeptides include proteins such as binding proteins, receptors, and antibodies. The terms “polypeptide” and “protein” are used interchangeably herein.

As used herein, the terms “providing,” “administering,” and “introducing” are used interchangeably herein and refer to the placement into a subject by a method or route which results in at least partial localization to a desired site.

### 2. Antiviral Hairpin Constructs

Disclosed herein are RNA hairpin constructs comprising a stem region having one or more polynucleotide sequences, wherein each of the one or more polynucleotide sequences individually has at least 70% (e.g., at least 75%, at least 80%, at least 85%, at least 90%, at least 95%, at least 96%, at least 97%, at least 98%, at least 99% or 100%) identity to a sequence from a target virus. The RNA hairpin constructs are configured for use in RNAi using RNA degradation pathways endogenous to plants and other eukaryotic systems. For example, the RNA hairpin may be targeted for degradation in a plant, e.g., processed using endogenous cellular machinery into an RNA silencing compound or compounds.

The target virus may be one or more orthotospoviruses. Exemplary orthotospoviruses include, but are not limited to: Alstroemeria necrotic streak orthotospovirus; Alstroemeria yellow spot orthotospovirus; Bean necrotic mosaic orthotospovirus; Calla lily chlorotic spot orthotospovirus; Capsicum chlorosis orthotospovirus; Chrysanthemum stem necrosis orthotospovirus; Groundnut bud necrosis orthotospovirus; Groundnut chlorotic fan spot orthotospovirus; Groundnut ringspot orthotospovirus; Groundnut yellow spot orthotospovirus; Hippcastrum chlorotic ringspot orthotospovirus; Impatiens necrotic spot orthotospovirus; Iris yellow spot orthotospovirus; Melon severe mosaic orthotospovirus; Melon yellow spot orthotospovirus; Mulberry vein banding associated orthotospovirus; Pepper chlorotic spot orthotospovirus; Polygonum ringspot orthotospovirus; Soybean vein necrosis orthotospovirus; Tomato chlorotic spot orthotospovirus; Tomato spotted wilt orthotospovirus; Tomato yellow ring orthotospovirus; Tomato zonate spot orthotospovirus; Watermelon bud necrosis orthotospovirus; Watermelon silver mottle orthotospovirus; and Zucchini lethal chlorosis orthotospovirus. In addition, both intraspecies and interspecies genomic reassortment has been documented between orthotospoviruses, further expanding upon its capacity to generate genetic diversity.

In select embodiments, the target virus is tomato spotted wilt virus (TSWV), tomato chlorotic spot virus (TCSV), groundnut ringspot virus (GRSV), impatiens necrotic spot virus (INSV), iris yellow spot virus (IYSV), melon severe mosaic virus (MeSMV), chrysanthemum stem necrosis virus (CSNV), soybean vein necrosis virus (SVNV), or a combination thereof.

In select embodiments, the orthotospovirus is a resistance-breaking orthotospovirus.

In some embodiments, the target virus is from a conserved region of the target virus genome. For example, conserved regions include those with regions of at least 25 nucleotides in which at least 85% (e.g., at least 90%, at least 95%, at least 96%, at least 97%, at least 98%, at least 99%, or 100%) of the nucleotide positions are conserved (e.g., between different target virus strains). In some embodiments, the conserved regions include at least two regions of at least 25 nucleotides in which at least 85% of the nucleotide positions are conserved.

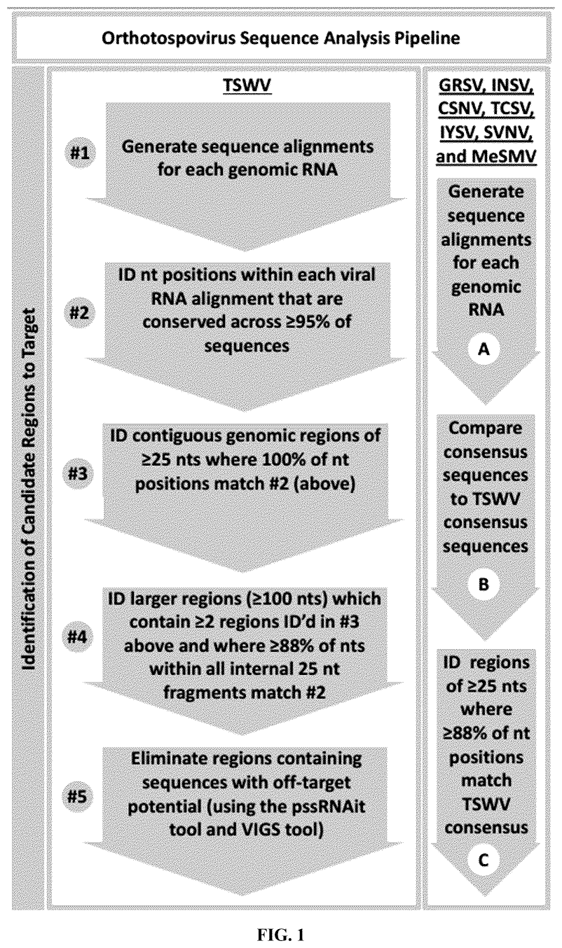

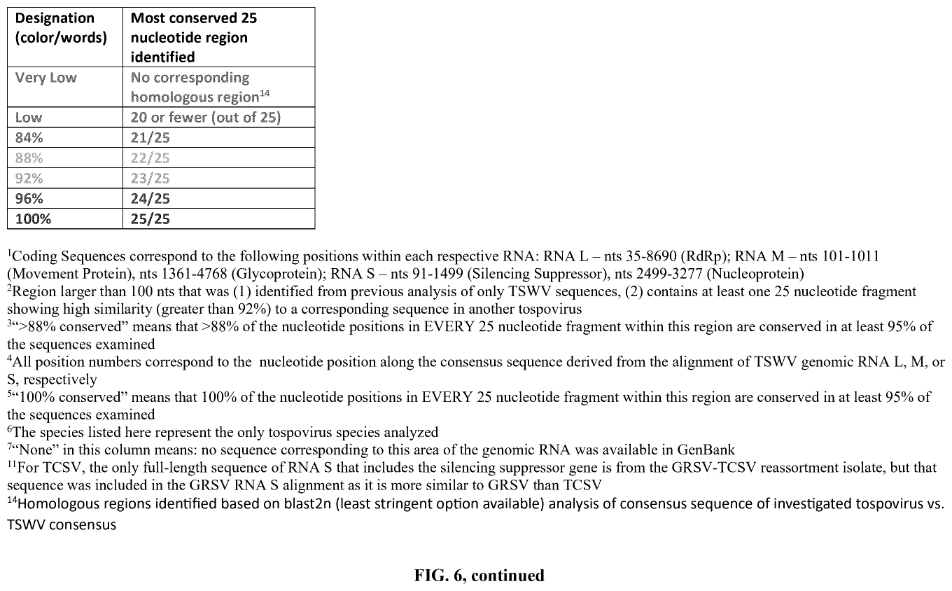

Tables 1 and 2 show the conserved regions which were identified for tomato spotted wilt virus (TSWV), and those considered for use in RNA hairpin constructs. In some embodiments, conserved regions include those regions conserved not only across strains of a single virus but also related viruses, e.g., viruses within the same family or genus (FIG. 6). See FIG. 1 and the Examples below for additional characteristics of the conserved regions.

In some embodiments, the conserved regions include sequences from the target virus which are transcribed into viral mRNA. For example, the conserved regions may include sequences from the target virus genome which are in a protein-encoding region. In some embodiments, the conserved regions include sequences which is in a domain which controls protein function.

In some embodiments, the RNA hairpin may comprise one or more polynucleotide sequences individually having at least 70% (e.g., at least 75%, at least 80%, at least 85%, at least 90%, at least 95%, at least 96%, at least 97%, at least 98%, at least 99% or 100%) identity to a sequence from a different gene of the target virus. In select embodiments, the hairpin may include one or more sequences having at least 70% (e.g., at least 75%, at least 80%, at least 85%, at least 90%, at least 95%, at least 96%, at least 97%, at least 98%, at least 99% or 100%) identity to each of the known genes and/or open reading frames of the target virus.

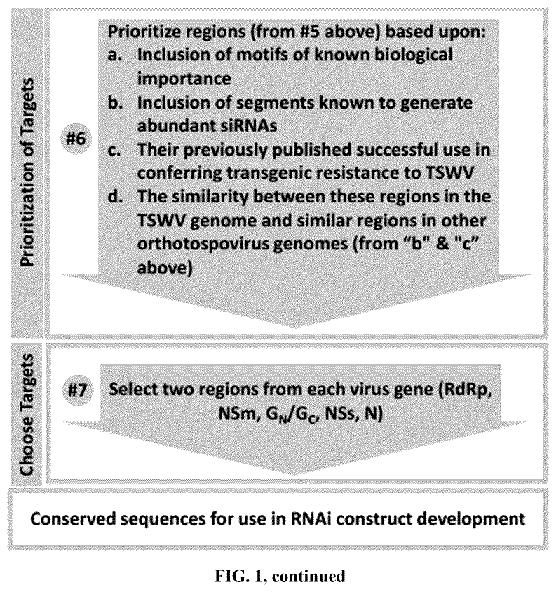

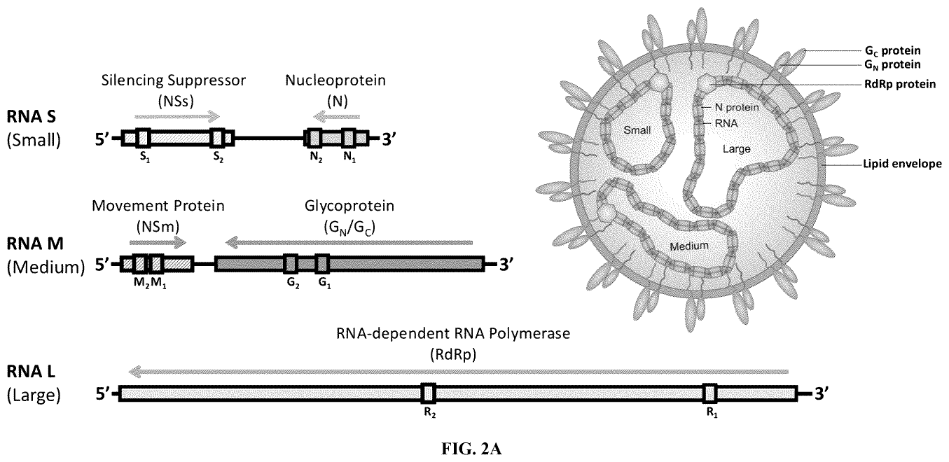

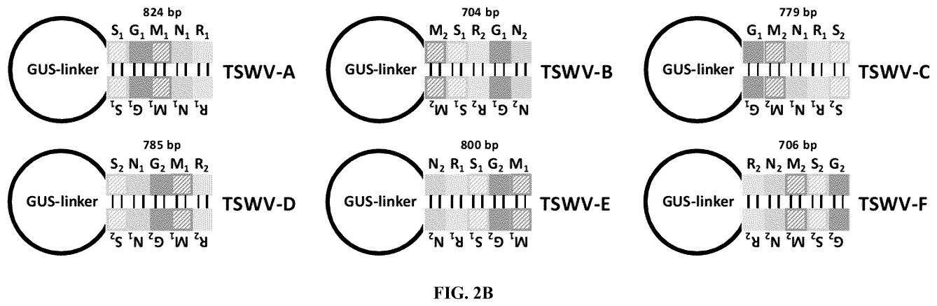

The (-) ssRNA viral genome of orthotospoviruses consists of three genomic RNAs and five viral genes. Genomic RNA L (8.9 kb for TSWV) encodes the viral RNA-dependent RNA-polymerase (RdRP). Genomic RNA M (4.8 kb for TSWV) encodes the NSm and glycoprotein (GN/GC), while the RNA S (2.9 kb for TSWV), encodes the viral non-structural silencing suppressor (NSs) and nucleocapsid (N). Since the genome is replicated by an RdRP without proofreading activity, these viruses, like other RNA viruses, have a high capacity for nucleotide diversity. For example, in tomato spotted wilt virus (TSWV), the hairpin may comprise a polynucleotide sequence individually having at least 70% identity to a sequence from the genes or open reading frames for: the silencing suppressor (NSs), the nucleoprotein (N), the movement protein (NSm), the glycoprotein (Gx/Gc), and the RNA-dependent RNA polymerase (RdRp). Scc FIG. 2, for example.

The conserved regions may also contain sequences which minimize potential off-target effects of RNA interference. Off-target effects in RNAi occur when the expression of genes other than the one in which the sequence was designed to target are modulated due to some level of complementarity of the off-target sequence with the RNAi molecule. Thus, the conserved regions may also comprise sequences which show little binding to endogenous plant sequences, thereby maximizing the specificity to the target virus sequence.

Each sequence having at least 70% identity to a sequence from a target virus may be of any length, e.g., 20-500 nucleotides. The sequence may be about 20-500 nucleotides, about 20-400 nucleotides, about 20-300 nucleotides, about 20-200 nucleotides, about 20-100 nucleotides, about 20-50 nucleotides, about 50-500 nucleotides, about 50-400 nucleotides, about 50-300 nucleotides, about 50-200 nucleotides, about 50-100 nucleotides, about 100-500 nucleotides, about 100-400 nucleotides, about 100-300 nucleotides, about 100-200 nucleotides, about 150-500 nucleotides, about 150-400 nucleotides, about 150-300 nucleotides, about 150-200 nucleotides, about 200-500 nucleotides, about 200-400 nucleotides, about 200-300 nucleotides, about 300-500 nucleotides, about 300-400 nucleotides, or about 400-500 nucleotides, In some embodiments, the length of each sequence is greater than 50 nucleotides, 100 nucleotides, 150 nucleotides, 200 nucleotides, 250 nucleotides, or more.

In some embodiments, the RNA hairpin comprises one or more polynucleotide sequences selected from SEQ ID NOs: 1-10. In select embodiments, the RNA hairpin comprises one of SEQ ID NOs: 1 or 2, one of SEQ ID NOs: 3 or 4, one of SEQ ID NOs: 5 or 6, one of SEQ ID NOs: 7 or 8, and/or one of SEQ ID NOs: 9 or 10. In select embodiments, the RNA hairpin comprises one of SEQ ID NOs: 1 or 2, one of SEQ ID NOs: 3 or 4, one of SEQ ID NOs: 5 or 6, one of SEQ ID NOs: 7 or 8, and one of SEQ ID NOs: 9 or 10. For example, the RNA hairpin may comprise SEQ ID NOs: 1, 3, 5, 7, and 9; 2, 4, 6, 8, and 10; 2, 3, 6, 8, and 9; 2, 4, 5, 7, and 10; 1, 3, 6, 7, and 10; and 1, 4, 5, 8, and 9.

R1 (SEQ ID NO: 1):
AAAGCAAAACAGTATACTAGAGATTTAGAAGGTTTCAAAGAAATACTGC
TCATGCTTAAATATTCTCTGATAAATGATGCATCAGGATTTAAAAGCTA
TAGAGCCACTGGAATGCATGCTGTTGAACTAATGGCAAAAAAGCACATA
GAGATAGGGGAATTCAACTT.
R2 (SEQ ID NO: 2):
AAAATGTCACCGGGTCTGTAGATTTCCTAGTTTCTGTTTTTGAGAAAAT
GCAGAGAACCAAAACAGACAGAGAAATATACTTGATGAGCATGAAAGTG
AAAATGATGCTTTATTTTATAGAGCA.
M1 (SEQ ID NO: 3):
GTCATCAAGATCTGTCCAACTTGGGACAGCAGAAAACAATACATGATGA
TTTCCAGGATTGTGATATGGGTCTGCCCCACTATACCAAACCCTACAGG
GAAACTTGTGGTTGCCCTGGTTGATCCCAACATGCCATCTGAAAAGCAA
GTCATTCT.
M2 (SEQ ID NO: 4):
CATCTTCCTTTGGAACCTATGAATCTGATTCTATCACAGAATCAGAGGG
TTATGATCTTTCTGCAAGAATGATAGTAGATACAAACCATCATATCTCA
AACTGGAAAAATGATCTTTTTGTTGGCAACGGGAAGCA.
G1 (SEQ ID NO: 5):
ATATTCCGACAGAATCCGCATGTAGCACCTTCATTAATTGCAAAACACC
AAGCTTCTTCACATCCCCAATAAGAAGTTGGTGTTATACAAAAATCTTG
GAAACCTGTTAAAGCTTGAT.
G2 (SEQ ID NO: 6):
ATTGATTTTTTCCCTATTGCTGCACAATCCCAAGACATGTCATCTCCTT
CTAGAGTTTTCTTAGTAAAAATAGGCACTCCATCATGAGTCAATTGTGG
GTGACCAAACATTTTCACAGGATCATTCAAGTTTGCAATATTTCCAGAA
TAA.
S1 (SEQ ID NO: 7):
CATGTGTATATCCCTATTTTTGATGATATTGATTTTAGCATCAATATTG
ATGACTCTGTTCTGGCACTATCTGTTTGCTCAAATACAGTCAATGCTAA
CGGAGTGAAACATCAAGGTCATTTGAAGGTTTTGTCTCCTGCTCAGCTC
CACTCTATTGGATCTA.
S2 (SEQ ID NO: 8):
CCTACAAAAGGAAATATTTCCTTTCAAAAACACTTGAATGTCTTCCATC
TAACACACAAACTATGTCTTATTTAGACAGCATCCAAATTCCTTCCTGG
AAGATAGACTTTGCCAGGGGAGAAATTAAAATTTC.
N1 (SEQ ID NO: 9):
GGAAGCAATCTTAGATTTGATAGTATTGAGATTCTCAGAATTCCCAGTT
TCCTCAACAAGCCTGACCCTGATCAAGCTATCAAGCCTTCTGAAGGTCA
TGTCAGTGGCTCCAATCCTGTCTGAAGTTTTCTTTATGGTAATTTTACC
AAAAGTAAAATCACTTT.
N2 (SEQ ID NO: 10):
TGCCTGTTTTTTAACCCCAAACATTTCATAGAACTTGTTAAGAGTTTCA
CTGTAATGTTCCATAGCAACACTTCCTTTAGCATTAGGATTGCTGGAAC
TAAGTAT.

The individual sequences from the one or more polynucleotide sequences (e.g., SEQ ID NO: 1-10) may be arranged in any order in the stem region of the RNA hairpin. As such the RNA hairpins are not limited by the order in which the one or more polynucleotide sequences are arranged. In some embodiments, each of the individual sequences is concatenated with those sequences with which it is adjacent. In some embodiments, each of the individual sequences may be separated by one or more (e.g., 1, 2, 3, 4, 5, 6, 7, 8, 9, 10, or more) nucleotides from the adjacent sequence. In some embodiments, the individual sequences are directly adjacent to the other sequence.

In some embodiments, the RNA hairpin comprises any of SEQ ID NOs: 11-16, or a variant having at least 70% identity to at least any of SEQ ID NOs: 11-16.

SEQ ID NO: 11 (824 nucleotides):
GGCCGTCAAGGCCACCGGTCATGTGTATATCCCTATTTTTGATGATATT
GATTTTAGCATCAATATTGATGACTCTGTTCTGGCACTATCTGTTTGCT
CAAATACAGTCAATGCTAACGGAGTGAAACATCAAGGTCATTTGAAGGT
TTTGTCTCCTGCTCAGCTCCACTCTATTGGATCTACTAGTATATTCCGA
CAGAATCCGCATGTAGCACCTTCATTAATTGCAAAACACCAAGCTTCTT
CACATCCCCAATAAGAAGTTGGTGTTATACAAAAATCTTGGAAACCTGT
TAAAGCTTGATCGATGTCATCAAGATCTGTCCAACTTGGGACAGCAGAA
AACAATACATGATGATTTCCAGGATTGTGATATGGGTCTGCCCCACTAT
ACCAAACCCTACAGGGAAACTTGTGGTTGCCCTGGTTGATCCCAACATG
CCATCTGAAAAGCAAGTCATTCTCCTAGGAAGCAATCTTAGATTTGATA
GTATTGAGATTCTCAGAATTCCCAGTTTCCTCAACAAGCCTGACCCTGA
TCAAGCTATCAAGCCTTCTGAAGGTCATGTCAGTGGCTCCAATCCTGTC
TGAAGTTTTCTTTATGGTAATTTTACCAAAAGTAAAATCACTTTCATAT
GAAAGCAAAACAGTATACTAGAGATTTAGAAGGTTTCAAAGAAATACTG
CTCATGCTTAAATATTCTCTGATAAATGATGCATCAGGATTTAAAAGCT
ATAGAGCCACTGGAATGCATGCTGTTGAACTAATGGCAAAAAAGCACAT
AGAGATAGGGGAATTCAACTTGTCGACGGCCTCATGGGCC.
SEQ ID NO: 12 (704 nucleotides):
GGCCGTCAAGGCCACCGGTCATCTTCCTTTGGAACCTATGAATCTGATT
CTATCACAGAATCAGAGGGTTATGATCTTTCTGCAAGAATGATAGTAGA
TACAAACCATCATATCTCAAACTGGAAAAATGATCTTTTTGTTGGCAAC
GGGAAGCACTAGTCATGTGTATATCCCTATTTTTGATGATATTGATTTT
AGCATCAATATTGATGACTCTGTTCTGGCACTATCTGTTTGCTCAAATA
CAGTCAATGCTAACGGAGTGAAACATCAAGGTCATTTGAAGGTTTTGTC
TCCTGCTCAGCTCCACTCTATTGGATCTATCGATAAAATGTCACCGGGT
CTGTAGATTTCCTAGTTTCTGTTTTTGAGAAAATGCAGAGAACCAAAAC
AGACAGAGAAATATACTTGATGAGCATGAAAGTGAAAATGATGCTTTAT
TTTATAGAGCACCTAGGATATTCCGACAGAATCCGCATGTAGCACCTTC
ATTAATTGCAAAACACCAAGCTTCTTCACATCCCCAATAAGAAGTTGGT
GTTATACAAAAATCTTGGAAACCTGTTAAAGCTTGATCATATGCCTGTT
TTTTAACCCCAAACATTTCATAGAACTTGTTAAGAGTTTCACTGTAATG
TTCCATAGCAACACTTCCTTTAGCATTAGGATTGCTGGAACTAAGTATG
TCGACGGCCTCATGGGCC.
SEQ ID NO: 13 (779 nucleotides):
GGCCGTCAAGGCCACCGGTATATTCCGACAGAATCCGCATGTAGCACCT
TCATTAATTGCAAAACACCAAGCTTCTTCACATCCCCAATAAGAAGTTG
GTGTTATACAAAAATCTTGGAAACCTGTTAAAGCTTGATACTAGTCATC
TTCCTTTGGAACCTATGAATCTGATTCTATCACAGAATCAGAGGGTTAT
GATCTTTCTGCAAGAATGATAGTAGATACAAACCATCATATCTCAAACT
GGAAAAATGATCTTTTTGTTGGCAACGGGAAGCATCGATGGAAGCAATC
TTAGATTTGATAGTATTGAGATTCTCAGAATTCCCAGTTTCCTCAACAA
GCCTGACCCTGATCAAGCTATCAAGCCTTCTGAAGGTCATGTCAGTGGC
TCCAATCCTGTCTGAAGTTTTCTTTATGGTAATTTTACCAAAAGTAAAA
TCACTTTCCTAGGAAAGCAAAACAGTATACTAGAGATTTAGAAGGTTTC
AAAGAAATACTGCTCATGCTTAAATATTCTCTGATAAATGATGCATCAG
GATTTAAAAGCTATAGAGCCACTGGAATGCATGCTGTTGAACTAATGGC
AAAAAAGCACATAGAGATAGGGGAATTCAACTTCATATGCCTACAAAAG
GAAATATTTCCTTTCAAAAACACTTGAATGTCTTCCATCTAACACACAA
ACTATGTCTTATTTAGACAGCATCCAAATTCCTTCCTGGAAGATAGACT
TTGCCAGGGGAGAAATTAAAATTTCGTCGACGGCCTCATGGGCC.
SEQ ID NO: 14 (785 nucleotides):
GGCCGTCAAGGCCACCGGTCCTACAAAAGGAAATATTTCCTTTCAAAAA
CACTTGAATGTCTTCCATCTAACACACAAACTATGTCTTATTTAGACAG
CATCCAAATTCCTTCCTGGAAGATAGACTTTGCCAGGGGAGAAATTAAA
ATTTCACTAGTGGAAGCAATCTTAGATTTGATAGTATTGAGATTCTCAG
AATTCCCAGTTTCCTCAACAAGCCTGACCCTGATCAAGCTATCAAGCCT
TCTGAAGGTCATGTCAGTGGCTCCAATCCTGTCTGAAGTTTTCTTTATG
GTAATTTTACCAAAAGTAAAATCACTTTATCGATTGATTTTTTCCCTAT
TGCTGCACAATCCCAAGACATGTCATCTCCTTCTAGAGTTTTCTTAGTA
AAAATAGGCACTCCATCATGAGTCAATTGTGGGTGACCAAACATTTTCA
CAGGATCATTCAAGTTTGCAATATTTCCAGAATAACCTAGGTCATCAAG
ATCTGTCCAACTTGGGACAGCAGAAAACAATACATGATGATTTCCAGGA
TTGTGATATGGGTCTGCCCCACTATACCAAACCCTACAGGGAAACTTGT
GGTTGCCCTGGTTGATCCCAACATGCCATCTGAAAAGCAAGTCATTCTC
ATATGAAAATGTCACCGGGTCTGTAGATTTCCTAGTTTCTGTTTTTGAG
AAAATGCAGAGAACCAAAACAGACAGAGAAATATACTTGATGAGCATGA
AAGTGAAAATGATGCTTTATTTTATAGAGCAGTCGACGGCCTCATGGGC
C.
SEQ ID NO: 15 (800 nucleotides):
GGCCGTCAAGGCCACCGGTGCCTGTTTTTTAACCCCAAACATTTCATAG
AACTTGTTAAGAGTTTCACTGTAATGTTCCATAGCAACACTTCCTTTAG
CATTAGGATTGCTGGAACTAAGTATACTAGTAAAGCAAAACAGTATACT
AGAGATTTAGAAGGTTTCAAAGAAATACTGCTCATGCTTAAATATTCTC
TGATAAATGATGCATCAGGATTTAAAAGCTATAGAGCCACTGGAATGCA
TGCTGTTGAACTAATGGCAAAAAAGCACATAGAGATAGGGGAATTCAAC
TTATCGATCATGTGTATATCCCTATTTTTGATGATATTGATTTTAGCAT
CAATATTGATGACTCTGTTCTGGCACTATCTGTTTGCTCAAATACAGTC
AATGCTAACGGAGTGAAACATCAAGGTCATTTGAAGGTTTTGTCTCCTG
CTCAGCTCCACTCTATTGGATCTACCTAGGATTGATTTTTTCCCTATTG
CTGCACAATCCCAAGACATGTCATCTCCTTCTAGAGTTTTCTTAGTAAA
AATAGGCACTCCATCATGAGTCAATTGTGGGTGACCAAACATTTTCACA
GGATCATTCAAGTTTGCAATATTTCCAGAATAACATATGTCATCAAGAT
CTGTCCAACTTGGGACAGCAGAAAACAATACATGATGATTTCCAGGATT
GTGATATGGGTCTGCCCCACTATACCAAACCCTACAGGGAAACTTGTGG
TTGCCCTGGTTGATCCCAACATGCCATCTGAAAAGCAAGTCATTCTGTC
GACGGCCTCATGGGCC.
SEQ ID NO: 16 (706 nucleotides):
GGCCGTCAAGGCCACCGGTAAAATGTCACCGGGTCTGTAGATTTCCTAG
TTTCTGTTTTTGAGAAAATGCAGAGAACCAAAACAGACAGAGAAATATA
CTTGATGAGCATGAAAGTGAAAATGATGCTTTATTTTATAGAGCACTAG
TTGCCTGTTTTTTAACCCCAAACATTTCATAGAACTTGTTAAGAGTTTC
ACTGTAATGTTCCATAGCAACACTTCCTTTAGCATTAGGATTGCTGGAA
CTAAGTATCGATCATCTTCCTTTGGAACCTATGAATCTGATTCTATCAC
AGAATCAGAGGGTTATGATCTTTCTGCAAGAATGATAGTAGATACAAAC
CATCATATCTCAAACTGGAAAAATGATCTTTTTGTTGGCAACGGGAAGC
ACCTAGGCCTACAAAAGGAAATATTTCCTTTCAAAAACACTTGAATGTC
TTCCATCTAACACACAAACTATGTCTTATTTAGACAGCATCCAAATTCC
TTCCTGGAAGATAGACTTTGCCAGGGGAGAAATTAAAATTTCATATGAT
TGATTTTTTCCCTATTGCTGCACAATCCCAAGACATGTCATCTCCTTCT
AGAGTTTTCTTAGTAAAAATAGGCACTCCATCATGAGTCAATTGTGGGT
GACCAAACATTTTCACAGGATCATTCAAGTTTGCAATATTTCCAGAATA
AGTCGACGGCCTCATGGGCC.

The RNA hairpin may further comprise a loop region having a single stranded linker sequence. The hairpin is not limited by the single stranded linker sequence. Generally, the single stranded linker sequence is from a sequence unrelated to the target virus or intended plant transformation host.

In some embodiments, the single stranded linker sequence is a fragment from the glucuronidase gene of E. coli. In select embodiments, the loop region comprises SEQ ID NO: 17.

SEQ ID NO: 17:
ATCTACCCGCTTCGCGTCGGCATCCGGTCAGTGGCAGTGAAGGGCGAAC
AGTTCCTGATTAACCACAAACCGTTCTACTTTACTGGCTTTGGTCGTCA
TGAAGATGCGGACTTGCGTGGCAAAGGATTCGATAACGTGCTGATGGTG
CACGACCACGCATTAATGGACTGGATTGGGGCCAACTCCTACCGTACCT
CGCATTACCCTTACGCTGAAGAGATGCTCGACTGGGCAGATGAACATGG
CATCGTGGTGATTGATGAAACTGCTGCTGTCGGCTTTAACCTCTCTTTA
GGCATTGGTTTCGAAGCGGGCAACAAGCCGAAAGAACTGTACAGCGAAG
AGGCAGTCAACGGGGAAACTCAGCAAGCGCACTTACAGGCGATTAAAGA
GCTGATAGCGCGTGACAAAAACCACCCAAGCGTGGTGATGTGGAGTATT
GCCAACGAACCGGATACCCGTCCGCAAGGTGCACGGGAATATTTCGCGC
CACTGGCGGAAGCAACGCGTAAACTCGACCCGACGCGTCCGATCACCTG
CGTCAATGTAATGTTCTGCGACGCTCACACCGATACCATCAGCGATCTC
TTTGATGTGCTGTGCCTGAACCGTTATTACGGATGGTATGTCCAAAGCG
GCGATTTGGAAACGGCAGAGAAGGTACTGGAAAAAGAACTTCTGGCCTG
GCAGGAGAAACTGCATCAGCCGATTATCATCACCGAATACGGCGTGGAT
ACGTTAGCCGGGCTGCACTCAATGTACACCGACATGTGGAGTGAAGAGT
ATCAGTGTGCATGGCTGGATATGTATCACCGCGTCTTTGATCGCGTCAG
CGCCGTCGTCGGTGAACAGGTATGGAATTTCGCCGATTTTGCGACCTCG
CAAGGCATATTGCGCGTTGGCGGTAACAAGAAAGGGATCTTCACTCG.

In some embodiments, the RNA hairpin comprises any of SEQ ID NOs: 18-23, or a variant having at least 70% identity to at least any of SEQ ID NOs: 18-23.

TSWV-A_Hairpin (SEQ ID NO: 18):
GCCCATGAGGCCGTCGACAAGTTGAATTCCCCTATCTCTATGTGCTTTT
TTGCCATTAGTTCAACAGCATGCATTCCAGTGGCTCTATAGCTTTTAAA
TCCTGATGCATCATTTATCAGAGAATATTTAAGCATGAGCAGTATTTCT
TTGAAACCTTCTAAATCTCTAGTATACTGTTTTGCTTTCATATGAAAGT
GATTTTACTTTTGGTAAAATTACCATAAAGAAAACTTCAGACAGGATTG
AGCCACTGACATGACCTTCAGAAGGCTTGATAGCTGTGATCAGGGTCAG
GCTTGTTGAGGAAACTGGGAATTCTGAGAATCTCAATACTATCAAATCT
AAGATTGCTTCCTAGGAGAATGACTTGCTTTTCAGATGGCATGTTGGGA
TCAACCAGGGCAACCACAAGTTTCCCTGTAGGGTTTGGTATAGTGGGGC
AGACCCATATCACAATCCTGGAAATCATCATGTATTGTTTTCTGCTGTC
CCAAGTTGGACAGATCTTGATGACATCGATCAAGCTTTAACAGGTTTCC
AAGATTTTTGTATAACACCAACTTCTTATTGGGGATGTGAAGAAGCTTG
GTGTTTTGCAATTAATGAAGGTGCTACATGCGGATTCTGTCGGAATATA
CTAGTAGATCCAATAGAGTGGAGCTGAGCAGGAGACAAAACCTTCAAAT
GACCTTGATGTTTCACTCCGTTAGCATTGACTGTATTTGAGCAAACAGA
TAGTGCCAGAACAGAGTCATCAATATTGATGCTAAAATCAATATCATCA
AAAATAGGGATATACACATGACCGGTGGCCTTGACGGCCCTTCTGCTTT
TTTGTACAAACTTGTGATGACGGTATCGATAAGCTTGATATCTACCCGC
TTCGCGTCGGCATCCGGTCAGTGGCAGTGAAGGGCGAACAGTTCCTGAT
TAACCACAAACCGTTCTACTTTACTGGCTTTGGTCGTCATGAAGATGCG
GACTTGCGTGGCAAAGGATTCGATAACGTGCTGATGGTGCACGACCACG
CATTAATGGACTGGATTGGGGCCAACTCCTACCGTACCTCGCATTACCC
TTACGCTGAAGAGATGCTCGACTGGGCAGATGAACATGGCATCGTGGTG
ATTGATGAAACTGCTGCTGTCGGCTTTAACCTCTCTTTAGGCATTGGTT
TCGAAGCGGGCAACAAGCCGAAAGAACTGTACAGCGAAGAGGCAGTCAA
CGGGGAAACTCAGCAAGCGCACTTACAGGCGATTAAAGAGCTGATAGCG
CGTGACAAAAACCACCCAAGCGTGGTGATGTGGAGTATTGCCAACGAAC
CGGATACCCGTCCGCAAGGTGCACGGGAATATTTCGCGCCACTGGCGGA
AGCAACGCGTAAACTCGACCCGACGCGTCCGATCACCTGCGTCAATGTA
ATGTTCTGCGACGCTCACACCGATACCATCAGCGATCTCTTTGATGTGC
TGTGCCTGAACCGTTATTACGGATGGTATGTCCAAAGCGGCGATTTGGA
AACGGCAGAGAAGGTACTGGAAAAAGAACTTCTGGCCTGGCAGGAGAAA
CTGCATCAGCCGATTATCATCACCGAATACGGCGTGGATACGTTAGCCG
GGCTGCACTCAATGTACACCGACATGTGGAGTGAAGAGTATCAGTGTGC
ATGGCTGGATATGTATCACCGCGTCTTTGATCGCGTCAGCGCCGTCGTC
GGTGAACAGGTATGGAATTTCGCCGATTTTGCGACCTCGCAAGGCATAT
TGCGCGTTGGCGGTAACAAGAAAGGGATCTTCACTCGATCGAATTCCTG
CAGCCCGGGGGATCCACTAGATGCATGCTCGAGCGGCCGCCAGTGTGAT
GGATATCTGCAGAATTCGCCCTTATCACAAGTTTGTACAAAAAAGCAGA
AGGGCCGTCAAGGCCACCGGTCATGTGTATATCCCTATTTTTGATGATA
TTGATTTTAGCATCAATATTGATGACTCTGTTCTGGCACTATCTGTTTG
CTCAAATACAGTCAATGCTAACGGAGTGAAACATCAAGGTCATTTGAAG
GTTTTGTCTCCTGCTCAGCTCCACTCTATTGGATCTACTAGTATATTCC
GACAGAATCCGCATGTAGCACCTTCATTAATTGCAAAACACCAAGCTTC
TTCACATCCCCAATAAGAAGTTGGTGTTATACAAAAATCTTGGAAACCT
GTTAAAGCTTGATCGATGTCATCAAGATCTGTCCAACTTGGGACAGCAG
AAAACAATACATGATGATTTCCAGGATTGTGATATGGGTCTGCCCCACT
ATACCAAACCCTACAGGGAAACTTGTGGTTGCCCTGGTTGATCCCAACA
TGCCATCTGAAAAGCAAGTCATTCTCCTAGGAAGCAATCTTAGATTTGA
TAGTATTGAGATTCTCAGAATTCCCAGTTTCCTCAACAAGCCTGACCCT
GATCAAGCTATCAAGCCTTCTGAAGGTCATGTCAGTGGCTCCAATCCTG
TCTGAAGTTTTCTTTATGGTAATTTTACCAAAAGTAAAATCACTTTCAT
ATGAAAGCAAAACAGTATACTAGAGATTTAGAAGGTTTCAAAGAAATAC
TGCTCATGCTTAAATATTCTCTGATAAATGATGCATCAGGATTTAAAAG
CTATAGAGCCACTGGAATGCATGCTGTTGAACTAATGGCAAAAAAGCAC
ATAGAGATAGGGGAATTCAACTTGTCGACGGCCTCATGGGC.
TSWV-B_Hairpin (SEQ ID NO: 19):
GCCCATGAGGCCGTCGACATACTTAGTTCCAGCAATCCTAATGCTAAAG
GAAGTGTTGCTATGGAACATTACAGTGAAACTCTTAACAAGTTCTATGA
AATGTTTGGGGTTAAAAAACAGGCATATGATCAAGCTTTAACAGGTTTC
CAAGATTTTTGTATAACACCAACTTCTTATTGGGGATGTGAAGAAGCTT
GGTGTTTTGCAATTAATGAAGGTGCTACATGCGGATTCTGTCGGAATAT
CCTAGGTGCTCTATAAAATAAAGCATCATTTTCACTTTCATGCTCATCA
AGTATATTTCTCTGTCTGTTTTGGTTCTCTGCATTTTCTCAAAAACAGA
AACTAGGAAATCTACAGACCCGGTGACATTTTATCGATAGATCCAATAG
AGTGGAGCTGAGCAGGAGACAAAACCTTCAAATGACCTTGATGTTTCAC
TCCGTTAGCATTGACTGTATTTGAGCAAACAGATAGTGCCAGAACAGAG
TCATCAATATTGATGCTAAAATCAATATCATCAAAAATAGGGATATACA
CATGACTAGTGCTTCCCGTTGCCAACAAAAAGATCATTTTTCCAGTTTG
AGATATGATGGTTTGTATCTACTATCATTCTTGCAGAAAGATCATAACC
CTCTGATTCTGTGATAGAATCAGATTCATAGGTTCCAAAGGAAGATGAC
CGGTGGCCTTGACGGCCCTTCTGCTTTTTTGTACAAACTTGTGATGACG
GTATCGATAAGCTTGATATCTACCCGCTTCGCGTCGGCATCCGGTCAGT
GGCAGTGAAGGGCGAACAGTTCCTGATTAACCACAAACCGTTCTACTTT
ACTGGCTTTGGTCGTCATGAAGATGCGGACTTGCGTGGCAAAGGATTCG
ATAACGTGCTGATGGTGCACGACCACGCATTAATGGACTGGATTGGGGC
CAACTCCTACCGTACCTCGCATTACCCTTACGCTGAAGAGATGCTCGAC
TGGGCAGATGAACATGGCATCGTGGTGATTGATGAAACTGCTGCTGTCG
GCTTTAACCTCTCTTTAGGCATTGGTTTCGAAGCGGGCAACAAGCCGAA
AGAACTGTACAGCGAAGAGGCAGTCAACGGGGAAACTCAGCAAGCGCAC
TTACAGGCGATTAAAGAGCTGATAGCGCGTGACAAAAACCACCCAAGCG
TGGTGATGTGGAGTATTGCCAACGAACCGGATACCCGTCCGCAAGGTGC
ACGGGAATATTTCGCGCCACTGGCGGAAGCAACGCGTAAACTCGACCCG
ACGCGTCCGATCACCTGCGTCAATGTAATGTTCTGCGACGCTCACACCG
ATACCATCAGCGATCTCTTTGATGTGCTGTGCCTGAACCGTTATTACGG
ATGGTATGTCCAAAGCGGCGATTTGGAAACGGCAGAGAAGGTACTGGAA
AAAGAACTTCTGGCCTGGCAGGAGAAACTGCATCAGCCGATTATCATCA
CCGAATACGGCGTGGATACGTTAGCCGGGCTGCACTCAATGTACACCGA
CATGTGGAGTGAAGAGTATCAGTGTGCATGGCTGGATATGTATCACCGC
GTCTTTGATCGCGTCAGCGCCGTCGTCGGTGAACAGGTATGGAATTTCG
CCGATTTTGCGACCTCGCAAGGCATATTGCGCGTTGGCGGTAACAAGAA
AGGGATCTTCACTCGATCGAATTCCTGCAGCCCGGGGGATCCACTAGAT
GCATGCTCGAGCGGCCGCCAGTGTGATGGATATCTGCAGAATTCGCCCT
TATCACAAGTTTGTACAAAAAAGCAGAAGGGCCGTCAAGGCCACCGGTC
ATCTTCCTTTGGAACCTATGAATCTGATTCTATCACAGAATCAGAGGGT
TATGATCTTTCTGCAAGAATGATAGTAGATACAAACCATCATATCTCAA
ACTGGAAAAATGATCTTTTTGTTGGCAACGGGAAGCACTAGTCATGTGT
ATATCCCTATTTTTGATGATATTGATTTTAGCATCAATATTGATGACTC
TGTTCTGGCACTATCTGTTTGCTCAAATACAGTCAATGCTAACGGAGTG
AAACATCAAGGTCATTTGAAGGTTTTGTCTCCTGCTCAGCTCCACTCTA
TTGGATCTATCGATAAAATGTCACCGGGTCTGTAGATTTCCTAGTTTCT
GTTTTTGAGAAAATGCAGAGAACCAAAACAGACAGAGAAATATACTTGA
TGAGCATGAAAGTGAAAATGATGCTTTATTTTATAGAGCACCTAGGATA
TTCCGACAGAATCCGCATGTAGCACCTTCATTAATTGCAAAACACCAAG
CTTCTTCACATCCCCAATAAGAAGTTGGTGTTATACAAAAATCTTGGAA
ACCTGTTAAAGCTTGATCATATGCCTGTTTTTTAACCCCAAACATTTCA
TAGAACTTGTTAAGAGTTTCACTGTAATGTTCCATAGCAACACTTCCTT
TAGCATTAGGATTGCTGGAACTAAGTATGTCGACGGCCTCATGGGC.
TSWV-C_Hairpin (SEQ ID NO: 20):
GGCCCATGAGGCCGTCGACGAAATTTTAATTTCTCCCCTGGCAAAGTCT
ATCTTCCAGGAAGGAATTTGGATGCTGTCTAAATAAGACATAGTTTGTG
TGTTAGATGGAAGACATTCAAGTGTTTTTGAAAGGAAATATTTCCTTTT
GTAGGCATATGAAGTTGAATTCCCCTATCTCTATGTGCTTTTTTGCCAT
TAGTTCAACAGCATGCATTCCAGTGGCTCTATAGCTTTTAAATCCTGAT
GCATCATTTATCAGAGAATATTTAAGCATGAGCAGTATTTCTTTGAAAC
CTTCTAAATCTCTAGTATACTGTTTTGCTTTCCTAGGAAAGTGATTTTA
CTTTTGGTAAAATTACCATAAAGAAAACTTCAGACAGGATTGGAGCCAC
TGACATGACCTTCAGAAGGCTTGATAGCTTGATCAGGGTCAGGCTTGTT
GAGGAAACTGGGAATTCTGAGAATCTCAATACTATCAAATCTAAGATTG
CTTCCATCGATGCTTCCCGTTGCCAACAAAAAGATCATTTTTCCAGTTT
GAGATATGATGGTTTGTATCTACTATCATTCTTGCAGAAAGATCATAAC
CCTCTGATTCTGTGATAGAATCAGATTCATAGGTTCCAAAGGAAGATGA
CTAGTATCAAGCTTTAACAGGTTTCCAAGATTTTTGTATAACACCAACT
TCTTATTGGGGATGTGAAGAAGCTTGGTGTTTTGCAATTAATGAAGGTG
CTACATGCGGATTCTGTCGGAATATACCGGTGGCCTTGACGGCCCTTCT
GCTTTTTTGTACAAACTTGTGATGACGGTATCGATAAGCTTGATATCTA
CCCGCTTCGCGTCGGCATCCGGTCAGTGGCAGTGAAGGGCGAACAGTTC
CTGATTAACCACAAACCGTTCTACTTTACTGGCTTTGGTCGTCATGAAG
ATGCGGACTTGCGTGGCAAAGGATTCGATAACGTGCTGATGGTGCACGA
CCACGCATTAATGGACTGGATTGGGGCCAACTCCTACCGTACCTCGCAT
TACCCTTACGCTGAAGAGATGCTCGACTGGGCAGATGAACATGGCATCG
TGGTGATTGATGAAACTGCTGCTGTCGGCTTTAACCTCTCTTTAGGCAT
TGGTTTCGAAGCGGGCAACAAGCCGAAAGAACTGTACAGCGAAGAGGCA
GTCAACGGGGAAACTCAGCAAGCGCACTTACAGGCGATTAAAGAGCTGA
TAGCGCGTGACAAAAACCACCCAAGCGTGGTGATGTGGAGTATTGCCAA
CGAACCGGATACCCGTCCGCAAGGTGCACGGGAATATTTCGCGCCACTG
GCGGAAGCAACGCGTAAACTCGACCCGACGCGTCCGATCACCTGCGTCA
ATGTAATGTTCTGCGACGCTCACACCGATACCATCAGCGATCTCTTTGA
TGTGCTGTGCCTGAACCGTTATTACGGATGGTATGTCCAAAGCGGCGAT
TTGGAAACGGCAGAGAAGGTACTGGAAAAAGAACTTCTGGCCTGGCAGG
AGAAACTGCATCAGCCGATTATCATCACCGAATACGGCGTGGATACGTT
AGCCGGGCTGCACTCAATGTACACCGACATGTGGAGTGAAGAGTATCAG
TGTGCATGGCTGGATATGTATCACCGCGTCTTTGATCGCGTCAGCGCCG
TCGTCGGTGAACAGGTATGGAATTTCGCCGATTTTGCGACCTCGCAAGG
CATATTGCGCGTTGGCGGTAACAAGAAAGGGATCTTCACTCGATCGAAT
TCCTGCAGCCCGGGGGATCCACTAGATGCATGCTCGAGCGGCCGCCAGT
GTGATGGATATCTGCAGAATTCGCCCTTATCACAAGTTTGTACAAAAAA
CAATCTTAGATTTGATAGTATTGAGATTCTCAGAATTCCCAGTTTCCTC
AGCAGAAGGGCCGTCAAGGCCACCGGTATATTCCGACAGAATCCGCATG
TAGCACCTTCATTAATTGCAAAACACCAAGCTTCTTCACATCCCCAATA
AGAAGTTGGTGTTATACAAAAATCTTGGAAACCTGTTAAAGCTTGATAC
TAGTCATCTTCCTTTGGAACCTATGAATCTGATTCTATCACAGAATCAG
AGGGTTATGATCTTTCTGCAAGAATGATAGTAGATACAAACCATCATAT
CTCAAACTGGAAAAATGATCTTTTTGTTGGCAACGGGAAGCATCGATGG
AAGACAAGCCTGACCCTGATCAAGCTATCAAGCCTTCTGAAGGTCATGT
CAGTGGCTCCAATCCTGTCTGAAGTTTTCTTTATGGTAATTTTACCAAA
AGTAAAATCACTTTCCTAGGAAAGCAAAACAGTATACTAGAGATTTAGA
AGGTTTCAAAGAAATACTGCTCATGCTTAAATATTCTCTGATAAATGAT
GCATCAGGATTTAAAAGCTATAGAGCCACTGGAATGCATGCTGTTGAAC
TAATGGCAAAAAAGCACATAGAGATAGGGGAATTCAACTTCATATGCCT
ACAAAAGGAAATATTTCCTTTCAAAAACACTTGAATGTCTTCCATCTAA
CACACAAACTATGTCTTATTTAGACAGCATCCAAATTCCTTCCTGGAAG
ATAGACTTTGCCAGGGGAGAAATTAAAATTTCGTCGACGGCCT
CATGGGCC.
TSWV-D_Hairpin (SEQ ID NO: 21):
GGCCCATGAGGCCGTCGACTGCTCTATAAAATAAAGCATCATTTTCACT
TTCATGCTCATCAAGTATATTTCTCTGTCTGTTTTGGTTCTCTGCATTT
TCTCAAAAACAGAAACTAGGAAATCTACAGACCCGGTGACATTTTCATA
TGAGAATGACTTGCTTTTCAGATGGCATGTTGGGATCAACCAGGGCAAC
CACAAGTTTCCCTGTAGGGTTTGGTATAGTGGGGCAGACCCATATCACA
ATCCTGGAAATCATCATGTATTGTTTTCTGCTGTCCCAAGTTGGACAGA
TCTTGATGACCTAGGTTATTCTGGAAATATTGCAAACTTGAATGATCCT
GTGAAAATGTTTGGTCACCCACAATTGACTCATGATGGAGTGCCTATTT
TTACTAAGAAAACTCTAGAAGGAGATGACATGTCTTGGGATTGTGCAGC
AATAGGGAAAAAATCAATCGATAAAGTGATTTTACTTTTGGTAAAATTA
CCATAAAGAAAACTTCAGACAGGATTGGAGCCACTGACATGACCTTCAG
AAGGCTTGATAGCTTGATCAGGGTCAGGCTTGTTGAGGAAACTGGGAAT
TCTGAGAATCTCAATACTATCAAATCTAAGATTGCTTCCACTAGTGAAA
TTTTAATTTCTCCCCTGGCAAAGTCTATCTTCCAGGAAGGAATTTGGAT
GCTGTCTAAATAAGACATAGTTTGTGTGTTAGATGGAAGACATTCAAGT
GTTTTTGAAAGGAAATATTTCCTTTTGTAGGACCGGTGGCCTTGACGGC
CCTTCTGCTTTTTTGTACAAACTTGTGATGACGGTATCGATAAGCTTGA
TATCTACCCGCTTCGCGTCGGCATCCGGTCAGTGGCAGTGAAGGGCGAA
CAGTTCCTGATTAACCACAAACCGTTCTACTTTACTGGCTTTGGTCGTC
ATGAAGATGCGGACTTGCGTGGCAAAGGATTCGATAACGTGCTGATGGT
GCACGACCACGCATTAATGGACTGGATTGGGGCCAACTCCTACCGTACC
TCGCATTACCCTTACGCTGAAGAGATGCTCGACTGGGCAGATGAACATG
GCATCGTGGTGATTGATGAAACTGCTGCTGTCGGCTTTAACCTCTCTTT
AGGCATTGGTTTCGAAGCGGGCAACAAGCCGAAAGAACTGTACAGCGAA
GAGGCAGTCAACGGGGAAACTCAGCAAGCGCACTTACAGGCGATTAAAG
AGCTGATAGCGCGTGACAAAAACCACCCAAGCGTGGTGATGTGGAGTAT
TGCCAACGAACCGGATACCCGTCCGCAAGGTGCACGGGAATATTTCGCG
CCACTGGCGGAAGCAACGCGTAAACTCGACCCGACGCGTCCGATCACCT
GCGTCAATGTAATGTTCTGCGACGCTCACACCGATACCATCAGCGATCT
CTTTGATGTGCTGTGCCTGAACCGTTATTACGGATGGTATGTCCAAAGC
GGCGATTTGGAAACGGCAGAGAAGGTACTGGAAAAAGAACTTCTGGCCT
GGCAGGAGAAACTGCATCAGCCGATTATCATCACCGAATACGGCGTGGA
TACGTTAGCCGGGCTGCACTCAATGTACACCGACATGTGGAGTGAAGAG
TATCAGTGTGCATGGCTGGATATGTATCACCGCGTCTTTGATCGCGTCA
GCGCCGTCGTCGGTGAACAGGTATGGAATTTCGCCGATTTTGCGACCTC
GCAAGGCATATTGCGCGTTGGCGGTAACAAGAAAGGGATCTTCACTCGA
TCGAATTCCTGCAGCCCGGGGGATCCACTAGATGCATGCTCGAGCGGCC
GCCAGTGTGATGGATATCTGCAGAATTCGCCCTTATCACAAGTTTGTAC
AAAAAAGCAGAAGGGCCGTCAAGGCCACCGGTCCTACAAAAGGAAATAT
TTCCTTTCAAAAACACTTGAATGTCTTCCATCTAACACACAAACTATGT
CTTATTTAGACAGCATCCAAATTCCTTCCTGGAAGATAGACTTTGCCAG
GGGAGAAATTAAAATTTCACTAGTGGAAGCAATCTTAGATTTGATAGTA
TTGAGATTCTCAGAATTCCCAGTTTCCTCAACAAGCCTGACCCTGATCA
AGCTATCAAGCCTTCTGAAGGTCATGTCAGTGGCTCCAATCCTGTCTGA
AGTTTTCTTTATGGTAATTTTACCAAAAGTAAAATCACTTTATCGATTG
ATTTTTTCCCTATTGCTGCACAATCCCAAGACATGTCATCTCCTTCTAG
AGTTTTCTTAGTAAAAATAGGCACTCCATCATGAGTCAATTGTGGGTGA
CCAAACATTTTCACAGGATCATTCAAGTTTGCAATATTTCCAGAATAAC
CTAGGTCATCAAGATCTGTCCAACTTGGGACAGCAGAAAACAATACATG
ATGATTTCCAGGATTGTGATATGGGTCTGCCCCACTATACCAAACCCTA
CAGGGAAACTTGTGGTTGCCCTGGTTGATCCCAACATGCCATCTGAAAA
GCAAGTCATTCTCATATGAAAATGTCACCGGGTCTGTAGATTTCCTAGT
TTCTGTTTTTGAGAAAATGCAGAGAACCAAAACAGACAGAGAAATATAC
TTGATGAGCATGAAAGTGAAAATGATGCTTTATTTTATAGAGC
AGTCGACGGCCTCATGGGCC.
TSWV-E_Hairpin (SEQ ID NO: 22):
GCCCATGAGGCCGTCGACAGAATGACTTGCTTTTCAGATGGCATGTTGG
GATCAACCAGGGCAACCACAAGTTTCCCTGTAGGGTTTGGTATAGTGGG
GCAGACCCATATCACAATCCTGGAAATCATCATGTATTGTTTTCTGCTG
TCCCAAGTTGGACAGATCTTGATGACATATGTTATTCTGGAAATATTGC
AAACTTGAATGATCCTGTGAAAATGTTTGGTCACCCACAATTGACTCAT
GATGGAGTGCCTATTTTTACTAAGAAAACTCTAGAAGGAGATGACATGT
CTTGGGATTGTGCAGCAATAGGGAAAAAATCAATCCTAGGTAGATCCAA
TAGAGTGGAGCTGAGCAGGAGACAAAACCTTCAAATGACCTTGATGTTT
CACTCCGTTAGCATTGACTGTATTTGAGCAAACAGATAGTGCCAGAACA
GAGTCATCAATATTGATGCTAAAATCAATATCATCAAAAATAGGGATAT
ACACATGATCGATAAGTTGAATTCCCCTATCTCTATGTGCTTTTTTGCC
ATTAGTTCAACAGCATGCATTCCAGTGGCTCTATAGCTTTTAAATCCTG
ATGCATCATTTATCAGAGAATATTTAAGCATGAGCAGTATTTCTTTGAA
ACCTTCTAAATCTCTAGTATACTGTTTTGCTTTACTAGTATACTTAGTT
CCAGCAATCCTAATGCTAAAGGAAGTGTTGCTATGGAACATTACAGTGA
AACTCTTAACAAGTTCTATGAAATGTTTGGGGTTAAAAAACAGGCACCG
GTGGCCTTGACGGCCCTTCTGCTTTTTTGTACAAACTTGTGATGACGGT
ATCGATAAGCTTGATATCTACCCGCTTCGCGTCGGCATCCGGTCAGTGG
CAGTGAAGGGCGAACAGTTCCTGATTAACCACAAACCGTTCTACTTTAC
TGGCTTTGGTCGTCATGAAGATGCGGACTTGCGTGGCAAAGGATTCGAT
AACGTGCTGATGGTGCACGACCACGCATTAATGGACTGGATTGGGGCCA
ACTCCTACCGTACCTCGCATTACCCTTACGCTGAAGAGATGCTCGACTG
GGCAGATGAACATGGCATCGTGGTGATTGATGAAACTGCTGCTGTCGGC
TTTAACCTCTCTTTAGGCATTGGTTTCGAAGCGGGCAACAAGCCGAAAG
AACTGTACAGCGAAGAGGCAGTCAACGGGGAAACTCAGCAAGCGCACTT
ACAGGCGATTAAAGAGCTGATAGCGCGTGACAAAAACCACCCAAGCGTG
GTGATGTGGAGTATTGCCAACGAACCGGATACCCGTCCGCAAGGTGCAC
GGGAATATTTCGCGCCACTGGCGGAAGCAACGCGTAAACTCGACCCGAC
GCGTCCGATCACCTGCGTCAATGTAATGTTCTGCGACGCTCACACCGAT
ACCATCAGCGATCTCTTTGATGTGCTGTGCCTGAACCGTTATTACGGAT
GGTATGTCCAAAGCGGCGATTTGGAAACGGCAGAGAAGGTACTGGAAAA
AGAACTTCTGGCCTGGCAGGAGAAACTGCATCAGCCGATTATCATCACC
GAATACGGCGTGGATACGTTAGCCGGGCTGCACTCAATGTACACCGACA
TGTGGAGTGAAGAGTATCAGTGTGCATGGCTGGATATGTATCACCGCGT
CTTTGATCGCGTCAGCGCCGTCGTCGGTGAACAGGTATGGAATTTCGCC
GATTTTGCGACCTCGCAAGGCATATTGCGCGTTGGCGGTAACAAGAAAG
GGATCTTCACTCGATCGAATTCCTGCAGCCCGGGGGATCCACTAGATGC
ATGCTCGAGCGGCCGCCAGTGTGATGGATATCTGCAGAATTCGCCCTTA
TCACAAGTTTGTACAAAAAAGCAGAAGGGCCGTCAAGGCCACCGGTGCC
TGTTTTTTAACCCCAAACATTTCATAGAACTTGTTAAGAGTTTCACTGT
AATGTTCCATAGCAACACTTCCTTTAGCATTAGGATTGCTGGAACTAAG
TATACTAGTAAAGCAAAACAGTATACTAGAGATTTAGAAGGTTTCAAAG
AAATACTGCTCATGCTTAAATATTCTCTGATAAATGATGCATCAGGATT
TAAAAGCTATAGAGCCACTGGAATGCATGCTGTTGAACTAATGGCAAAA
AAGCACATAGAGATAGGGGAATTCAACTTATCGATCATGTGTATATCCC
TATTTTTGATGATATTGATTTTAGCATCAATATTGATGACTCTGTTCTG
GCACTATCTGTTTGCTCAAATACAGTCAATGCTAACGGAGTGAAACATC
AAGGTCATTTGAAGGTTTTGTCTCCTGCTCAGCTCCACTCTATTGGATC
TACCTAGGATTGATTTTTTCCCTATTGCTGCACAATCCCAAGACATGTC
ATCTCCTTCTAGAGTTTTCTTAGTAAAAATAGGCACTCCATCATGAGTC
AATTGTGGGTGACCAAACATTTTCACAGGATCATTCAAGTTTGCAATAT
TTCCAGAATAACATATGTCATCAAGATCTGTCCAACTTGGGACAGCAGA
AAACAATACATGATGATTTCCAGGATTGTGATATGGGTCTGCCCCACTA
TACCAAACCCTACAGGGAAACTTGTGGTTGCCCTGGTTGATCCCAACAT
GCCATCTGAAAAGCAAGTCATTCTGTCGACGGCCTCATGGGC.
TSWV-F_Hairpin (SEQ ID NO: 23):
GCCCATGAGGCCGTCGACTTATTCTGGAAATATTGCAAACTTGAATGAT
CCTGTGAAAATGTTTGGTCACCCACAATTGACTCATGATGGAGTGCCTA
TTTTTACTAAGAAAACTCTAGAAGGAGATGACATGTCTTGGGATTGTGC
AGCAATAGGGAAAAAATCAATCATATGAAATTTTAATTTCTCCCCTGGC
AAAGTCTATCTTCCAGGAAGGAATTTGGATGCTGTCTAAATAAGACATA
GTTTGTGTGTTAGATGGAAGACATTCAAGTGTTTTTGAAAGGAAATATT
TCCTTTTGTAGGCCTAGGTGCTTCCCGTTGCCAACAAAAAGATCATTTT
TCCAGTTTGAGATATGATGGTTTGTATCTACTATCATTCTTGCAGAAAG
ATCATAACCCTCTGATTCTGTGATAGAATCAGATTCATAGGTTCCAAAG
GAAGATGATCGATACTTAGTTCCAGCAATCCTAATGCTAAAGGAAGTGT
TGCTATGGAACATTACAGTGAAACTCTTAACAAGTTCTATGAAATGTTT
GGGGTTAAAAAACAGGCAACTAGTGCTCTATAAAATAAAGCATCATTTT
CACTTTCATGCTCATCAAGTATATTTCTCTGTCTGTTTTGGTTCTCTGC
ATTTTCTCAAAAACAGAAACTAGGAAATCTACAGACCCGGTGACATTTT
ACCGGTGGCCTTGACGGCCCTTCTGCTTTTTTGTACAAACTTGTGATGA
CGGTATCGATAAGCTTGATATCTACCCGCTTCGCGTCGGCATCCGGTCA
GTGGCAGTGAAGGGCGAACAGTTCCTGATTAACCACAAACCGTTCTACT
TTACTGGCTTTGGTCGTCATGAAGATGCGGACTTGCGTGGCAAAGGATT
CGATAACGTGCTGATGGTGCACGACCACGCATTAATGGACTGGATTGGG
GCCAACTCCTACCGTACCTCGCATTACCCTTACGCTGAAGAGATGCTCG
ACTGGGCAGATGAACATGGCATCGTGGTGATTGATGAAACTGCTGCTGT
CGGCTTTAACCTCTCTTTAGGCATTGGTTTCGAAGCGGGCAACAAGCCG
AAAGAACTGTACAGCGAAGAGGCAGTCAACGGGGAAACTCAGCAAGCGC
ACTTACAGGCGATTAAAGAGCTGATAGCGCGTGACAAAAACCACCCAAG
CGTGGTGATGTGGAGTATTGCCAACGAACCGGATACCCGTCCGCAAGGT
GCACGGGAATATTTCGCGCCACTGGCGGAAGCAACGCGTAAACTCGACC
CGACGCGTCCGATCACCTGCGTCAATGTAATGTTCTGCGACGCTCACAC
CGATACCATCAGCGATCTCTTTGATGTGCTGTGCCTGAACCGTTATTAC
GGATGGTATGTCCAAAGCGGCGATTTGGAAACGGCAGAGAAGGTACTGG
AAAAAGAACTTCTGGCCTGGCAGGAGAAACTGCATCAGCCGATTATCAT
CACCGAATACGGCGTGGATACGTTAGCCGGGCTGCACTCAATGTACACC
GACATGTGGAGTGAAGAGTATCAGTGTGCATGGCTGGATATGTATCACC
GCGTCTTTGATCGCGTCAGCGCCGTCGTCGGTGAACAGGTATGGAATTT
CGCCGATTTTGCGACCTCGCAAGGCATATTGCGCGTTGGCGGTAACAAG
AAAGGGATCTTCACTCGATCGAATTCCTGCAGCCCGGGGGATCCACTAG
ATGCATGCTCGAGCGGCCGCCAGTGTGATGGATATCTGCAGAATTCGCC
CTTATCACAAGTTTGTACAAAAAAGCAGAAGGGCCGTCAAGGCCACCGG
TAAAATGTCACCGGGTCTGTAGATTTCCTAGTTTCTGTTTTTGAGAAAA
TGCAGAGAACCAAAACAGACAGAGAAATATACTTGATGAGCATGAAAGT
GAAAATGATGCTTTATTTTATAGAGCACTAGTTGCCTGTTTTTTAACCC
CAAACATTTCATAGAACTTGTTAAGAGTTTCACTGTAATGTTCCATAGC
AACACTTCCTTTAGCATTAGGATTGCTGGAACTAAGTATCGATCATCTT
CCTTTGGAACCTATGAATCTGATTCTATCACAGAATCAGAGGGTTATGA
TCTTTCTGCAAGAATGATAGTAGATACAAACCATCATATCTCAAACTGG
AAAAATGATCTTTTTGTTGGCAACGGGAAGCACCTAGGCCTACAAAAGG
AAATATTTCCTTTCAAAAACACTTGAATGTCTTCCATCTAACACACAAA
CTATGTCTTATTTAGACAGCATCCAAATTCCTTCCTGGAAGATAGACTT
TGCCAGGGGAGAAATTAAAATTTCATATGATTGATTTTTTCCCTATTGC
TGCACAATCCCAAGACATGTCATCTCCTTCTAGAGTTTTCTTAGTAAAA
ATAGGCACTCCATCATGAGTCAATTGTGGGTGACCAAACATTTTCACAG
GATCATTCAAGTTTGCAATATTTCCAGAATAAGTCGACGGCCTCATGGG
C.

Also disclosed herein are nucleic acids encoding an RNA hairpin described herein. Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns).

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

To construct cells that express the present hairpins, expression vectors for stable or transient expression of the present system may be constructed via conventional methods and introduced into cells. For example, nucleic acids encoding the disclosed hairpins may be cloned into a suitable expression vector, such as a plasmid or a viral vector in operable linkage to a suitable promoter. The selection of expression vectors/plasmids/viral vectors should be suitable for integration and replication in eukaryotic cells.

In some embodiments, the nucleic acids may be adapted to use in plants. In one embodiment, a series of plant-specific vectors are provided for expression of the RNA hairpin in plants. The vectors may be optimized for transient expression of the RNA hairpin in plant protoplasts, or for stable integration and expression in intact plants via the Agrobacterium-mediated transformation. The vectors may be optimized for transient expression of the RNA hairpin in plant protoplasts, or for stable integration and expression in intact plants via the Agrobacterium-mediated transformation. In one aspect, the vector constructs include a nucleotide sequence comprising a DNA-dependent RNA polymerase III promoter, wherein the promoter is operably linked to a gRNA molecule and a Pol III terminator sequence, and a nucleotide sequence comprising a DNA-dependent RNA polymerase II promoter operably linked to a nucleic acid sequence encoding the RNA hairpin.

Further disclosed herein are compositions comprising an RNA hairpin described herein or a nucleic acid encoding thereof. The disclosed RNA hairpins may be incorporated into compositions that may be suitable for administration (e.g., to a plant). The compositions may include carriers. The term “carrier,” as used herein, means a non-toxic, inert solid, semi-solid or liquid filler, diluent, encapsulating material, surfactant, cyclodextrins or formulation auxiliary of any type. A carrier may include a single ingredient or a combination of two or more ingredients. Some examples of materials which can serve as carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, preservatives and antioxidants can also be present in the composition, according to the judgment of the formulator. Carriers typically include at least one of diluents, lubricants, binders, disintegrants, colorants, antioxidants, preservatives, glidants, solvents, suspending agents, wetting agents, surfactants, cyclodextrins combinations thereof, and others. All carriers are optional in the compositions.

Embodiments disclosed herein also include methods of generating an antiviral hairpin construct for use in increasing resistance to a target virus. The methods comprise identifying conserved region of a target virus genome; selecting sequences from the conserved regions which are in protein-encoding domains; compiling selected sequences for each gene or open reading frame of the target virus into a single concatenated sequence; and generating the antiviral hairpin construct with the following regions i) a first region comprising compiled sequences, ii) a second region comprising a linker sequence configured to form a loop, and iii) a third region complementary to the compiled sequences configured to form a double stranded stem with the first region. Descriptions of the conserved regions and selected sequences are the same as those provided above and found in FIG. 1 and in the Examples.

### 3. Methods

The present disclosure also provides methods for increasing virus resistance or preventing viral infection in a plant. The methods comprise providing an RNA hairpin or a nucleic acid encoding thereof, as described above, to the plant, or a plant cell, seed, fruit, plant part, or propagation material of the plant.

The term “plant propagation material” refers to generative parts of a plant, which can be used for the multiplication of the plant, and vegetative plant material such as cuttings and tubers (e.g., potatoes). In some embodiments, the propagation material is a root, a corm, a tuber, a bulb, a slip, a cutting of the plant, and a rhizome. Parts of a plant are any sections of a plant (e.g., roots, cotyledons, tendrils, leaves, flowers, seeds, stems, callus tissue, nuts, and fruit) that develop from a plant propagation material or grow at a later time. The methods described herein can be used on any plant part. Examples of plant parts include but are not limited to the root, corm, tuber, bulb, slip and rhizome.

In some embodiments, the virus is one or more orthotospoviruses. Exemplary orthotospoviruses include, but are not limited to: Alstroemeria necrotic streak virus; Alstroemeria yellow spot virus; Bean necrotic mosaic virus; Calla lily chlorotic spot virus; Capsicum chlorosis virus; Chrysanthemum stem necrosis virus; Groundnut bud necrosis virus; Groundnut chlorotic fan spot virus; Groundnut ringspot virus; Groundnut yellow spot virus; Hippeastrum chlorotic ringspot virus; Impatiens necrotic spot virus; Iris yellow spot virus; Melon severe mosaic virus; Melon yellow spot virus; Mulberry vein banding associated virus; Pepper chlorotic spot virus; Polygonum ringspot virus; Soybean vein necrosis virus; Tomato chlorotic spot virus; Tomato spotted wilt virus; Tomato yellow ring virus; Tomato zonate spot virus; Watermelon bud necrosis virus; Watermelon silver mottle virus; and Zucchini lethal chlorosis virus. In addition, both intraspecies and interspecies genomic reassortment has been documented between orthotospoviruses, further expanding upon its capacity to generate genetic diversity.

In select embodiments, the virus is tomato spotted wilt virus (TSWV), tomato chlorotic spot virus (TCSV), groundnut ringspot virus (GRSV), impatiens necrotic spot virus (INSV), iris yellow spot virus (IYSV), melon severe mosaic virus (MeSMV), chrysanthemum stem necrosis virus (CSNV), soybean vein necrosis virus (SVNV), or a combination thereof. In select embodiments, the orthotospovirus is a resistance-breaking orthotospovirus.

In some embodiments, the virus is Orthotospovirus alstroemerinecrosis; Orthotospovirus alstroemeriflavi; Orthotospovirus phaseolinecrotessellati; Orthotospovirus callaflavi; Orthotospovirus capsiciflavi; Orthotospovirus chrysanthinecrocaulis; Orthotospovirus arachinecrosis; Orthotospovirus arachiflavi; Orthotospovirus arachianuli; Orthotospovirus arachiflavamaculae; Orthotospovirus hippeflavi; Orthotospovirus impatiensnecromaculae; Orthotospovirus iridimaculaflavi; Orthotospovirus melotessellati; Orthotospovirus meloflavi; Orthotospovirus morivenae; Orthotospovirus capsicimaculaflavi; Orthotospovirus polygonianuli; Orthotospovirus glycininecrovenae; Orthotospovirus tomatoflavi; Orthotospovirus tomatomaculae; Orthotospovirus tomatanuli; Orthotospovirus tomatozonae; Orthotospovirus citrullonecrosis; Orthotospovirus citrullomaculosi; or Orthotospovirus cucurbichlorosis.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation.

The transformation can be transient or stable transformation. As used herein, the term “stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant. In select embodiments, the nucleic acid encoding the RNA hairpin may be stably integrated into the plant genome, for example via Agrobacterium-mediated transformation.

Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. Sec., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

As such, the disclosure also provides plants and plant propagation materials (e.g., plant cell, seed, fruit, or plant parts) produced using the methods disclosed herein. Genetically modified, transformed or transgenic plants include a plant into which an exogenous polynucleotide, e.g., a polynucleotide encoding an RNA hairpin as disclosed herein, has been introduced.

The methods disclosed herein are suitable for use with any plant, for example, grain crops, fruit crops, forage crops, root vegetable crops, leafy vegetable crops, flowering plants, conifers, trees, oil crops, plants used in phytoremediation, industrial crops, medicinal crops, laboratory model plants, and the like. As such, non-limiting examples of plants that may be used with the present methods include: grains, forage crops, fruits, vegetables, oil seed crops, palms, forestry, vines, maize (corn, Zea mays), banana, peanut, field peas, sunflower, tomato, peppers, canola, tobacco, wheat, barley, oats, potato, soybeans, cotton, carnations, sorghum, lupin, rice, rutabaga, celery, switchgrass, apple, petunias, Arabidopsis thaliana, Medicago truncatula, Medicago sativa, Brachypodium distachyon, Nicotiana benthamiana, or Setaria viridis.

Exemplary plants that can be treated using the methods disclosed herein include but are not limited to the following monocots and dicots: bulb vegetables; cereal grains (such as wheat, barley, rice; corn; citrus fruits (such as grapefruit, lemon, and orange); cotton and other fiber crops; cucurbits; fruiting vegetables; leafy vegetables (such as celery, head and leaf lettuce, and spinach); legumes (such as soybeans, green beans, chick peas, lentils); oil seed crops; peanut; pome fruit (such as apple and pear); stone fruits (such as almond, pecan, and walnut); root vegetables; tuber vegetables; corm vegetables; tobacco; strawberry and other berries; cole crops (such as broccoli, cabbage); grape; plants used for biomass production (such as miscanthus, bamboo); pineapple; flowering plants; bedding plants; grasses; and perennial plants, including plantation crops such as banana and coffee. The methods described herein are also suitable for ornamental plants. Ornamental plants include but are not limited to the plants which are grown in greenhouses, nurseries, urban and suburban greenspaces, landscaped areas and properties, home properties, parks, vertical farming, hydroponic operations, and hoop houses. The plants within the ornamental market are varied and broad, but generally contain non-turf and non-food plants primarily grown for aesthetic or utility purposes.

The present systems and methods can be used to transform a number of monocotyledonous and dicotyledonous plants and plant cell systems. Thus, the methods described herein can be utilized with dicotyledonous plants belonging, for example, to the orders Solanales, Magniolales, Illiciales, Laurales, Piperales, Aristochiales, Nymphacales, Ranunculales, Papeverales, Sarraceniaceac, Trochodendrales, Hamamelidales, Eucomis, Leitneriales, Myricales, Fagales, Casuarinaceae, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alscodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna: the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

In some embodiments, the plant is a food crop (such as peanuts, watermelons, capsicums, tomatoes, peppers, zucchinis, et al.) or ornamental crop (such as ornamental species which are important to flower farms, e.g., calla lily, impatiens, chrysanthemums, iris, et al.). In select embodiments, the plant is selected from: peppers, tobacco, peanuts, alliums, impatiens, petunias, and chrysanthemums.

In some embodiments, the methods further comprise providing another viral resistance treatment to the plant. Other viral resistance treatments include, but are not limited to, other genes conferring resistance to the virus (e.g., Sw genes), thrips transmission (transgenic expression of viral glycoprotein), and acyl sugars traits that provide protection against the thrips vector that translates into a reduction in virus transmission.

### 4. Kits

Embodiments of the present disclosure also provide kits or systems comprising an RNA hairpin or a nucleic acid encoding thereof, as disclosed herein, and a transfection or transformation reagent.

The kits can also comprise other agents and/or products co-packaged, co-formulated, and/or co-delivered with other components. The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein.

The kits provided herein are in suitable packaging. Suitable packaging includes, but is not limited to, vials, bottles, jars, flexible packaging, and the like. Individual member components of the kits may be physically packaged together or separately.

### 5. Examples

The accompanying Examples are offered as illustrative as a partial scope and particular embodiments of the disclosure and are not meant to be limiting of the scope of the disclosure.

### Materials and Methods

Bioinformatic pipeline to identify conserved target sequences of orthotospoviruses for hairpin construct designs. Orthotospovirus resistance constructs were designed based on existing TSWV, GRSV, INSV, CSNV, TCSV, IYSV, SVNV, and MeSMV sequence information and cloned into appropriate expression vectors for plant transformation. A bioinformatic pipeline was developed to include sequential steps towards prioritizing candidate target sequences (FIG. 1). Sequence information from each of the orthotospoviruses (listed) above was downloaded from GenBank on Jul. 16, 2014, and compiled into a fasta-formatted file. Alignments of all available sequence information for each respective orthotospovirus were generated using ClustalX v.2.0. In total, 24 alignments were generated (three genomic RNAs per orthotospovirus species by eight species) consisting of a total of 3,296 whole and partial orthotospovirus sequences. The program SeaView v.4.4.1 was used for alignment editing and generation of consensus sequences showing nucleotide positions with 90-95% base conservation among sequences analyzed. MEGA version 6 was used for visualization of consensus sequences and conversion of consensus alignments to Excel format. Microsoft Excel® 2010 was used to identify TSWV regions of 25 nucleotides or more where at least 88% of nucleotide positions are conserved in at least 95% of the sequences examined. Furthermore, 51% majority rule consensus sequences from TSWV corresponding to all three genomic RNAs were compared to similar consensus sequences from other orthotospovirus sequence alignments. Regions of 25 nucleotides or more where over 88% of nucleotide positions were conserved between TSWV and other orthotospoviruses were identified. Larger regions (of over 100 nucleotides) which included multiple identified regions of conservation based on the above criteria were chosen for further analysis. To screen the identified regions for their potential to have off-target effects in tomato, the pssRNAit tool available from The Samuel Roberts Noble Foundation (plantgrn.noble.org/pssRNAit/) and the VIGS tool available from the Sol Genomics Network (solgenomics.net/tools/vigs) were used. For off-target analysis, the default parameters for the pssRNAit tool were modified with the “Expect” parameter set to 1.5 and the “Off-target accessibility” parameter set to 20, and the plant genome used for off-target analysis was the tomato genome. Potential sequences within the regions of interest that included so-called “toxic motifs” which have the potential to have widespread negative effects on plant hosts when silenced were also identified with the pssRNAit tool and excluded from subsequent consideration. The default parameters for the VIGS tool were altered to identify potential off-target genes in tomato with less than 2 mismatches versus the identified region from TSWV. Further analysis was also carried out to identify the TSWV genomic region off-target potential versus the Honey Bee and Human Genomes using the RNAiScan tool from The Samuel Noble Roberts Foundation (bioinfo2.noble.org/RNAiScan.htm), with the default parameters changed to look for regions with 1 or fewer mismatches.

Four criteria were used to prioritize conserved target regions (FIG. 1, step 6): i) conserved sequence motifs within protein-encoding domains known to have indispensable functions (Table 7); ii) segments of TSWV sequence known to generate abundant siRNAs in infected plants; iii) genome segments from prior publications reporting transgenic resistance to orthotospoviruses; and iv) sequence similarity between regions identified by ii) and iii) and falling within the TSWV 51% consensus identified from the pipeline (FIG. 1, steps 1-5). Regions of orthotospovirus genomes previously identified to be capable of providing orthotospovirus resistance in host plants were given priority in choosing from amongst the pool of considered conserved regions for construct design.

Cloning of hairpin constructs. For each of the five genes of TSWV, two “best-candidate” conservation regions were identified (dsRNA targets) using the pipeline described above (FIG. 1, step 7) which maximized the conservation at the nucleotide level while minimizing any identified potential for off-target effects. These 10 candidate regions (Table 2) were selected to create six unique hairpin constructs, each including five genomic fragments from TSWV (one from each gene) (FIG. 2). Constructs were designed for Gateway® cloning and with unique restriction sites between each TSWV genomic fragment. Unique restriction sites were identified by comparison of the fragments with the vectors pENTR™ D-TOPOR (Life Technologies, Carlsbad, CA) and pENTRTM223.1 (Arabidopsis Biological Resource Center, Columbus, Ohio) using search tools NEBcutter v2.0 and RestrictionMapper v.3 (restrictionmapper.org). TSWV hairpin constructs consisting of two inverted, complimentary arms connected by a GUS linker (refer to FIG. 2)—a 926 nucleotide sequence fragment in sense orientation from the glucuronidase gene of E. coli-were synthesized via gBlocks® Gene Fragments (Integrated DNA Technologies, Coralville, Iowa, USA) and cloned into the Sfil sites of vector pENTRTM223.1 (stock #CD3-1741, Arabidopsis Biological Resource Center, Columbus, Ohio, USA). Constructs in pENTR223.1 were transferred via Gateway® LR Clonase® II (Thermo Fisher Scientific, Waltham, MA, USA) to the vector pANDA35HK (Miki and Shimamoto 2004).

To serve as a negative hairpin transgenic control, a 305 bp sequence of green fluorescent protein cDNA (short hairpin GFP, shGFP) was PCR-amplified from pSITE-2NB vector (Chakrabarty et al., 2007; GenBank accession: EF212296.1) using previously designed primers, cloned into pENTR/D-TOPO vector using a directional TOPO cloning as described above, and transformed into chemically competent E. coli cells (One Shot cells, Thermo Scientific, Wilmington, DE, USA). The length of the insert was determined by whole cell PCR and the sequence inserted was verified through DNA sequencing (GENEWIZ, South Plainfield, NJ, USA). Colonies of E. coli positive for the GFP hairpin insert were grown overnight in selective broth and plasmid was purified using standard miniprep protocol (Qiagen, Germantown, MD, USA). The insert in the pENTR/D-TOPO vector was then transferred to pANDA35HK as described above.

Plant transformation. Hairpin constructs in pANDA35HK were used to transform tomato (Solanum lycopersicum L.) ‘Moneymaker’ via Agro-mediated transformation with Agrobacterium tumefaciens (strain LBA4404). Plant transformations were carried out by The Ralph M. Parsons Foundation Plant Transformation Facility (University of California, Davis, CA) according to methods adapted from Fillatii et al. (Nat. Biotechnol. 5:726-730). Transformants possessing the nptll selectable marker were identified initially based upon their growth on media containing Kanamycin. Multiple lines were generated from each of the five constructs (A-F as described previously, the C construct was delayed in transformation, with only one transgenic event, and therefore not selected for virus screens).

Tomato propagation and seed extraction. Tomato plants were propagated under greenhouse conditions at Kansas State University with temperatures ranging from 25° C. (day) to 23° C. (night) with a day-light cycle of 16 h of light and 8 h of darkness. Tomatoes were potted in 1000 series (9.46 liters) pots containing MetroMix 900 potting soil (Sun Gro®, Agawam, MA, USA). Initially, soil was amended with Miracle-Gro Shake 'n Feed® Tomatoes, Fruit, and Vegetable Continuous Release Plant Food. Throughout the experiments, plants were fertilized weekly with Miracle-Gro® Liquafeed Tomato, Fruit, and Vegetables Plant Food. Subsequent generations were propagated from seeds extracted from collected tomato fruit, and seedlings were transplanted into 1000 series (9.46 liters) pots in the greenhouse at the 3-4 leaflet stage and maintained as described above. In later successions of whole-plant resistance screens, plants were propagated under growth chamber conditions at the NCSU Phytotron, a state-of-the-art, environmentally-controlled, plant-growing and experimentation facility. Plant were grown in a supplied mix of gravel (#16 construction grade, steam-sterilized) and peat-lite (Redi-Earth, Sun Gro Horticulture) in large pots (3.78 liters, 20.32 cm-diam. or 5.68 liters, 25.4 cm-diam.). To collect seeds, harvested fruit were crushed and kept in 1-gallon plastic zip bags or pulp was removed and stored in 50-ml plastic screw-cap centrifuge tubes, all kept at room temperature to promote fermentation and separation of seeds from pulp, and then seeds were poured over a sieve, rinsed in water, dried thoroughly on paper towels and placed in paper seed envelopes to be stored at 6° C.

Transgene verification. The presence of the transgene (DNA) and expression (RNA) of each construct was verified in each generation (TO, T1, T2, and T3) by PCR, and reverse transcription (RT)-PCR, respectively. Leaves were collected from young tomato plants (5-6 leaf stage for TO-T2, 2 leaf stage for T3) and DNA and RNA were extracted from leaflet samples using the DNeasy® Plant Mini Kit (Qiagen, Hilden, Germany) and RNeasy® Plant Mini Kit (Qiagen, Hilden, Germany), respectively; Genomic DNA Mini Kit (Plant) (IBI Scientific, IA, USA) was used for DNA extraction from T3 plants. For the TO-T2 leaf tissue, ˜ 100 mg of tissue was flash frozen in 1.7 ml microfuge tubes and ground by hand with a Kontes pestle (Thermo Fisher Scientific, Waltham, MA), and for T3 samples, one metal bead (Daisy Precision Max BB's, 0.177 Caliber BB Zinc Plated Steel, Walmart) was added to a compatible 2 ml screw-capped microfuge tube containing the frozen tissue and finely pulverized with the use of a TissueLyserII (Qiagen, Germantown, MD) for 2 minutes at 30.0 1/s frequency. Construct-specific (hairpin arms) and/or GUS linker primers were used to detect the transgene via polymerase chain reaction (PCR) using the following program: a 2-min heating step at 95° C. followed by 30 cycles of 30 sec melting at 95° C., 30 sec annealing at 50° C. or 55° C., and 1 min or 30 sec elongation at 72° C. with a final extension of 2 or 5 min at 72° C. cDNA was prepared from extracted RNA using the Verso cDNA Synthesis Kit (Thermo Fisher Scientific, Waltham, MA, USA). cDNA was then tested using the same primers according to the protocol described above for PCR. To determine whether the parent line was homozygous for the transgene, ten T2 seedlings were randomly selected from among 40 emergent seedlings and, after DNA extraction, each of the ten seedlings was tested for the presence of the transgene. If the transgene was present in all ten seedlings, it was concluded that the parent line was likely homozygous for the transgene.

Only plants (lineages) that tested positive for the presence of the transgene were moved forward to virus resistance screens using thrips inoculation-leaf disk assays in the laboratory, and whole-plant, mechanical rub inoculation assays performed in the growth chamber or greenhouse. Lineages that exhibited resistance were followed to the next generation of transgenic parents (TO-T3) for seed production.

Virus species and isolates. Three TSWV isolates were used in resistance screens over the course of the study: i) a Hawaiian isolate (TSWV-HI), originally collected from Maui (Ullman et al., 1992, Phytopathology 82:1087) and maintained in plant cultures for over 30 years, was used to challenge TO-T2 leaf tissue by thrips inoculation and T1 plants by mechanical, rub inoculation; ii) a Western North Carolina isolate (TSWV-NC) collected from a commercial fresh market tomato farm in 2020 and used to challenge T2 whole plants by mechanical inoculation; and iii) a resistance-breaking isolate (Shymanovich et al. 2023, in press), collected in 2020 from a processing tomato farm in Firebaugh, California (RB-TSWV-CA), maintained in plant culture in Sw-5-containing tomato varieties and confirmed to have the C118Y resistance-breaking mutation in the NSm gene (Batuman et al. 2017, Plant Dis. 101:637), was used to challenge T3 leaf tissue by thrips inoculation and whole plants by mechanical inoculation. A Florida tomato isolate of TCSV originally collected from Hendry County, FL, in 2012 and maintained in culture in pepper, jimsonweed (Datura stramonium) and tomato, was used to test T2 plants by mechanical inoculation.

Virus resistance screens via thrips inoculation. Experiments were conducted using an adapted leaf disk assay. Cohorts of age-synchronized young larvae (Lls) of Frankliniella occidentalis from a lab-reared colony originating from an isolate collected from Oahu, HI (Bautista et al., 1995, Phytopathology 85:953-958) were given 24-hour acquisition access periods (AAP) on TSWV-infected leaf tissue of Emilia sonchifolia (TSWV-HI, common lab strain) or Solanum lycopersicon cv. ‘Mountain Merit’ (RB-TSWV-CA strain), and then moved to green bean pods to rear to adulthood. Multiple plants per hairpin transgene (FIG. 2B: TSWV-A to F, and shGFP negative control) and non-transgenic controls [cv. ‘Moneymaker’ or ‘Mountain Merit’ (Sw-5b resistance gene; RB-TSWV test only)] served as inoculation hosts. Briefly, from these plants, three 15-mm-diameter leaf disks were cork-bored from different leaves per plant, then disks were singly placed inside 15 mL conical tubes with five to 10 adult thrips per disk, the tubes were sealed, and the thrips were allowed a 48-hour or 72-hour inoculation access period (IAP) for the TO-T2, and T3 generation screens, respectively. For the T3 screen, a 1 ml, tip-sealed pipet tip filled with water and covered with a Kimwipe tissue served as a moistened surface at the bottom of the conical tube upon which to place the leaf disk as a means to reduce moisture loss, and the tube was covered with a cap fitted with a thrips proof screen to allow for gas exchange. Thrips were removed after the IAP, and each leaf disks was incubated on 1 mL diH₂O in a well of a 24-well microplate for four days to allow viral replication. Leaf disk samples were removed and pulverized and virus infection status was determined by Double Antibody Sandwich Enzyme Linked Immuno-Sorbent Assay (DAS-ELISA) using the ELISA Reagent Set for tomato spotted wilt virus (Agdia, Elkhart, IN, USA) according to the manufacturer's instructions. Reactions were examined in 96-well microtiter plates and absorbance was measured at 405 nm using a Synergy H1 Hybrid Reader (Biotek, Winooski, VT, USA) or BioTek Cytation 5 Cell Imaging Multimode Reader (Agilent, CA, USA), for the TO-T2 and T3 screens, respectively. A plant was considered infected if at least one leaf disk (three excised per plant) tested positive for TSWV, and ‘resistance’ was defined in these assays as statistically significant reduction (P<0.05) in plant infection incidence of each test transgenic compared to the transgenic (shGFP) and non-transgenic controls.

Virus resistance screens via mechanical inoculation of plants. Progeny from T1 parent lines (e.g., T2 test plants) that exhibited resistance by thrips inoculation in the leaf disk assay were selected for whole plant resistance experiments under growth chamber and greenhouse conditions. Each experiment consisted of the five test lines (A-F transgenes), the transgenic negative control line (shGFP) and the non-transgenic background control (‘Moneymaker’) with 10-13 replicate plants per line and control in a randomized block design, with at least two replicates of each line and control per block. Each experiment was conducted three (TSWV-HI) or two times (TCSV), i.e., independent biological replicates, and the two virus species were tested in entirely separate experiments by different researchers (TSWV-HI at NCSU; TCSV at ARS, Fort Pierce, FL).

Ten-day-old seedlings of each line or control were transplanted into pots and positioned into the experiment blocks. Each plant was rub-inoculated at the time of transplant and a second time at 16 days-post-transplant to increase likelihood of virus infection. Virus-infected and symptomatic tomato (cv. ‘Moneymaker’) served as the inoculum source. Using standard virological methods, leaf tissue was ground in 10 mM sodium sulfite buffer with a cold mortar and pestle, carborundum powder was dusted on leaf surfaces of all expanded leaves per plant, and cotton swabs drenched in plant sap were used to rub-inoculate each leaf. While typical symptoms associated with host susceptibility, including leaf chlorosis and necrosis, chlorotic ring spots, leaf curling and rugosity, internode shortening and overall reduced stature were noted over the course of the experiments, plant height was chosen as the least biased and most consistent, quantitative measure of disease associated with these viruses. Four weeks after transplanting, plant heights were recorded and younger leaf tissues (not inoculated) were sampled and prepared for DAS-ELISA detection of TSWV or TCSV according to the manufacturer's specifications (Agdia). Each sample was prepared in two technical replicate plates to avoid potential contamination bias, and plants were considered virus-positive when absorbance values (wavelength=405 nm) were greater than 3 times the average absorbance values of the non-inoculated healthy controls.

Using the TSWV-NC isolate, progeny from T2 parent lines (e.g., T3 test plants) that exhibited resistance in the whole plant T2 screen were selected for screening in the greenhouse in five insect cages (0.6906 meter 1×0.6906 meter w×0.6906 meter h) (customized) (BioQuip Products Inc., CA, USA), with each cage comprising one block with the same experiment structure (transgenic test lines, shGFP negative control, non-transgenic controls) and line replication as described above, with the exception that multiple lineages of promising A, E, and F parents were included (7 lines in total), and a TSWV-resistant (Sw-5 R gene) tomato cultivar ‘Mountain Glory’ F1 (Harris Seeds, Rochester, NY, USA) was included as an additional non-transgenic, control with the expectation that it would be resistant to the TSWV-NC isolate. Plants were grown in MetroMix 900 potting soil (Sun Gro®, Agawam, MA, USA) and fertilized weekly with (Miracle-Gro® Tomato Plant Food, co, state, USA) from one week after emergence and continuously thereafter. The caged plants were placed under supplemental lights (12 hour photoperiod) and experienced approximately 26° C. day and 24° C. night temperatures (+/−3 degrees). The plants were screened for the presence of the transgene as described above to ensure T3 test plants entering the screen tested positive for the transgene prior to virus inoculation. Mechanical, leaf rub-inoculations were performed as described above. The TSWV-NC screen was conducted once, and leaf tissues were sampled 14 days post-inoculation for DAS-ELISA detection of TSWV using the BioTek Cytation 5 Cell Imaging Multimode Reader. Non-inoculated control plants (‘Moneymaker’ and ‘Mountain Glory’) housed in each cage served as ELISA-negative controls for the assay.

Based on the outcome of the TSWV-NC screen, the same seven T3 lines plus an additional E line, and the same three sets of controls (+/−TSWV) were mechanically inoculated with the RB-TSWV-CA isolate and housed in the greenhouse using the same growing conditions. Plants were placed in cages in a randomized block design with eight blocks (=same dimensions as Bioquip cages above) in the 1st trial of the experiment, and 4 blocks (=1.829 meters 1×1.829 meters w×1.829 meters h, BugDorm) (Megaview Science Company Ltd., Taichung, Taiwan) in the 2nd trial to accommodate the additional line and extended duration of the experiment. For both trials, plants were sampled at two time points (14 and 24 days-post-inoculation) for TSWV detection in younger, ‘systemic’ leaf tissue (non-inoculated, expanded leaves), and healthy, non-inoculated control tissues (“Moneymaker” and “Mountain Glory”) in each cage served as ELISA-negative controls. Plant heights were recorded at the termination of the experiments.

Transgene expression in T3 flowers and fruit. RNA was extracted from the outer pericarp (skin included) of fruit and whole flowers of five-month-old tomato plants to examine transgene expression in reproductive tissues. Leaf tissue from young, expanded leaves were also included in the analysis as positive RT-PCR controls. Leaf and flower tissues were pulverized with the TissucLyserIl as described above. For tomato fruit, roughly 1 cm² of outer pericarp was excised using a razor blade, cleaning the blade with deionized water and 70% ethanol between samples, flash-frozen, then ground in liquid N₂ using a mortar and pestle. RNA was extracted using the Mini Total RNA Kit for plants (IBI Scientific, IA, USA) followed by removal of contaminating DNA with a TURBO DNA-free™ Kit (Invitrogen, CA, USA). GUS linker primers were used to detect the transgene from the generated cDNA as described above. DNase-treated RNA template served as a negative expression control for PCR to ensure that only cDNA, and not residual DNA, was amplified.

Thrips inoculation assay on healthy, detached tomato fruit from T3 transgenics. Based on previous methodologies (Aramburu ct al, 2000, J. Phytopathol. 148 11-12:569-574), detached fruits were exposed to adult thrips infected with non-RB TSWV-NC or RB-TSWV-CA to determine if fruit from the transgenic plants express resistance to the virus. Several preliminary trials were conducted with the non-RB TSWV isolate and three controls [shGFP transgenic, ‘Moneymaker’ (no Sw-5b), and ‘Mountain Merit’ (Sw-5b)] to optimize the inoculation access period (IAP), symptom development, and fruit ripeness. The optimized experiment consisted of the three controls and the four, best-performing T3 plants (progeny of T2 parental lines) determined from the whole-plant virus resistance screen. Each TSWV isolate was screened in separate, independent experiments, and each experiment was conducted three times. For each control and transgenic line, green fruits just before ripening were collected. While attempts were made to standardize for green fruit size (time of inoculation), time to fruit-ripening tended to vary by cultivar and between transgenic lines. Each experimental unit consisted of five or six fruits in one thrips-inoculation arena—a plastic 16-ounce DeliPro container sealed with a lid fitted with thrips-proof screen (100 micron nylon mesh) for gas exchange, and a disc of filter paper at the bottom to absorb excess moisture. Cohorts of infected thrips were obtained as described above for the leaf disk assay, and adult infection rates were determined by normalized RT-qPCR (TSWV N RNA to thrips actin RNA).

Fifty viruliferous adults (1:1 female: male) were placed in each arena for a 5-day IAP at room temperature under light conditions. As healthy controls, aviruliferous thrips (same sex ratio) were placed in an arena containing one fruit from each control or transgenic line for each experiment for the purpose of visual comparison to virus treatment groups and serving as an ELISA negative control. A single control cup with a representative fruit from each group was used due to limited fruit availability. Observations were made daily for thrips exploration of the fruit, and feeding was documented by the presence of feeding scars. At the end of the IAP, sealed cups were placed in a 27-gallon plastic container with three Hot Shot No-Pest Insect Pest strips (˜67.7 ounces, 18% Dichlorvos (2,2-dichlorovinyl dimethyl phosphate)) (St. Louis, MO) and firmly sealed for 24 hours to kill the thrips. Cups were removed and left to incubate at room temperature for three weeks or until fruit threatened to over ripen. Two pericarp samples were collected from each fruit-one from either symptomatic or highly-scarred tissue and the second from non-symptomatic or lightly-scarred tissue. Each sample was placed into 2 ml Safelock microfuge tubes with two metal beads and ground using the TissueLyserII for 45 seconds to one minute at 30.0 1/s frequency. Virus infection status was determined via DAS-ELISA using the Agdia Reagent Set for TSWV (Agdia, Elkhart, IN, USA) in a 96-well microtiter plate format according to manufacturer's instructions. Each sample was plated as two technical well replicates, and each plate included at least one positive control, one ‘MoneyMaker’ leaf tissue-negative control (no virus), two no-virus, thrips-infested fruit negative controls, and general extract buffer (GEB) negative controls (3 wells). Absorbance was measured at 405 nm using a BioTek Cytation 5 Cell Imaging Multimode Reader (Agilent, CA, USA). Samples were considered TSWV-positive if ELISA values were at least two times the average absorbance values of the GEB negative controls.

Statistical analyses. Nonparametric and generalized linear model-fit statistical tests were performed to determine the efficacy of each transgenic line against shGFP (transgenic negative control) and nontransgenic negative controls of commercial varieties (‘MoneyMaker’ and ‘Mountain Merit’). All tests were performed using JMP Pro, v15 (SAS Institute, Cary, NC). For the leaf disk assay-TSWV resistance screens, Fisher's Exact Tests of Independence were performed on contingency tables (r×c) of plant infection status (yes, no) x line or variety, followed by pairwise exact tests between each transgenic line and negative control to determine significance (P<0.05) for each pedigree (or generation). For the whole plant experiment-virus resistance screens, infection incidence data (number of plants infected/total tested for each line or variety) were pooled across biological repetitions of each virus test (n=2 for TSWV-HI, n=3 for TCSV-FL, and n=2 for RB-TSWV-CA) for each line and variety, and the binary data (infection status=yes, no) was subjected to a generalized linear model (Firth-based adjusted estimates) using binomial distribution (link function=logit) to fit the data. Contrasts were performed between each transgenic line and shGFP, and between the various controls (shGFP, ‘Moneymaker’, ‘Mountain Merit’) to determine significant differences (P<0.05). In addition, plant height data from these experiments were also pooled accordingly and subjected to the Kruskal Wallis Wilcoxin method, followed by pairwise Tukey tests to determine statistical significance (P<0.05) between lines and controls.

### Example 1

### Hairpin Design

Using a bioinformatics pipeline, 13 conserved nucleotide regions (>80% conserved) ranging from 100 to 167 nucleotides in length across L (4 sequences), M (4 sequences) and S (5 sequences) genome segments were identified, which comprised a total of 31 highly conserved (100%) smaller regions (median length=32 nucleotides) across the five TSWV ORFs (Table 1). To further narrow the targets, regions of conservation identified among the different orthotospoviruses found in North America were prioritized and included in hairpin construct design (Table 2, FIG. 6) in different combinations and orders (FIG. 2). Lastly, the literature was also searched for descriptions of biological function for viral protein domains or amino acid residues critical for protein function (Table 2) in plant or insect hosts. For several of the conserved regions, it was identified that these conserved sequences also have well-established molecular roles. The TSWV GC protein has a fusion domain that is highly conserved for this species and among other members of this genus and the order Bunyavirales. One of the NSm target domains overlaps with the known ‘D-motif’, a conserved domain found in ‘30K superfamily’ virus movement proteins. Targeting conserved functional domains could produce more durable resistance to these viruses.

Table 1. Most conserved genomic regions of tomato spotted wilt virus identified.

| | | | TSWV Larger | Smaller |
|---|---|---|---|---|
| | | | Region¹ | Regions |
| | Genome | | (>88% | (100% |
| ID | RNA | Genomic Region | conserved²) | conserved)³ |
| R3 | L⁴ | RNA dependent RNA | 2639-2738⁵ (100)⁶ | 2668-2693 (26) |
| | | polymerase | | 2698-2729 (32) |
| R2 | L | RNA dependent RNA | 3851-3974 (124) | 3851-3875 (29) |
| | | polymerase | | 3910-3962 (53) |
| R1 | L | RNA dependent RNA | 7527-7693 (167) | 7527-7576 (50) |
| | | polymerase | | 7584-7612 (29) |
| | | | | 7635-7666 (31) |
| | | | | 7668-7693 (26) |
| R4 | L | RNA dependent RNA | 7846-7960 (115) | 7848-7876 (29) |
| | | polymerase | | 7916-7951 (36) |
| M2 | M | Movement protein | 291-427 (137) | 301-343 (43) |
| | | | | 357-418 (62) |
| M1 | M | Movement protein | 441-595 (155) | 444-493 (50) |
| | | | | 495-538 (44) |
| | | | | 561-592 (32) |
| G2 | M | Glycoprotein | 2184-2333 (150) | 2244-2269 (26) |
| | | | | 2298-2329 (32) |
| G1 | M | Glycoprotein | 2550-2667 (118) | 2580-2644 (65) |
| S1 | S | Silencing suppressor | 318-480 (163) | 329-358 (30) |
| | | | | 385-410 (26) |
| | | | | 417-442 (26) |
| | | | | 444-480 (37) |
| S3 | S | Silencing suppressor | 938-1096 (159) | 952-980 (29) |
| | | | | 1021-1093 (73) |
| S2 | S | Silencing suppressor | 1261-1393 (133) | 1265-1330 (66) |
| | | | | 1350-1375 (26) |
| N2 | S | Nucleoprotein | 2520-2625 (106) | 2539-2581 (43) |
| N1 | S | Nucleoprotein | 2909-3072 (164) | 2910-2962 (53) |
| | | | | 2979-3037 (59) |
| | | | | 3039-3070 (32) |

¹Region larger than 100 that was (1) identified from previous analysis of only TSWV sequences, (2) contains at least one 25 nucleotide fragment showing high similarity (greater than 92%) to a corresponding sequence in another tospovirus;
²“>88% conserved” means that >88% of the nucleotide positions in EVERY 25 nucleotide fragment within this region are conserved in at least 95% of the sequences examined;
³“100% conserved” means that 100% of the nucleotide positions in EVERY 25 nucleotide fragment within this region are conserved in at least 95% of the sequences examined;
⁴Coding sequences correspond to the following positions within each respective RNA: RNA L-35-8690 (RdRp); RNA M-101-1011 (Movement Protein), 1361-4768 (Glycoprotein); RNA S-91-1499 (Silencing Suppressor), 2499-3277 (Nucleoprotein);
⁵All position numbers correspond to the nucleotide position along the consensus sequence derived from the alignment of TSWV genomic RNA L, M, or S, respectively;
⁶Number in () = number of nucleotides in length.

Table 2. Candidate genomic regions of high conservation selected for transgene construct designs to target all genes of tomato spotted wilt virus and other orthotospovirus species.

| | | | Larger | Smaller | |
|---|---|---|---|---|---|
| | | Genomic | Region^{d} | Regions | |
| Region | Genome | Region | (>88% | (100% | Other |
| Name | RNA | (ORF)^{c} | conserved)^{e} | conserved)^{f} | Features |
| R2 | L | RdRP | 3851-3974^{g} | 3851-3875, | N/A^{h} |
| | | | | 3910-3962 | |
| R1 | L | RdRP | 7527-7693 | 7527-7576, | N/A |
| | | | | 7584-7612, | |
| | | | | 7635-7666, | |
| | | | | 7668-7693 | |
| M2 | M | NSm | 291-427 | 301-343, | P/D-L-X |
| | | | | 357-418 | Motif |
| | | | | | [402-407] |
| M1 | M | NSm | 441-595 | 444-493, | RB Change |
| | | | | 495-538, | Sites |
| | | | | 561-592 | [453-455], |
| | | | | | [459-461] |
| | | | | | D-Motif |
| | | | | | [561-563] |
| G2 | M | Glyco- | 2184-2333 | 2244-2269, | N/A |
| | | protein | | 2298-2329 | |
| G1 | M | Glyco- | 2550-2667 | 2580-2644 | Fusion |
| | | protein | | | Domain |
| | | | | | [2552-2617] |
| S1 | S | NSs | 318-480 | 329-358, | Avr |
| | | | | 385-410, | Determinant |
| | | | | 417-442, | (Tsw gene) |
| | | | | 444-480 | [unk] |
| S2 | S | NSs | 1261-1393 | 1265-1330, | Avr |
| | | | | 1350-1375 | Determinant |
| | | | | | (Tsw gene) |
| | | | | | [unk] |
| | | | | | YL Motif |
| | | | | | [1329-1334] |
| N2 | S | N | 2520-2625 | 2539-2581 | N/A |
| N1 | S | N | 2909-3072 | 2910-2962, | N/A |
| | | | | 2979-3037, | |
| | | | | 3039-3070 | |

^{c}Coding sequences correspond to the following positions within each respective RNA: L RNA-RNA-dependent RNA polymerase (RdRP) = nts 35-8690; M RNA-Movement protein (NSm) = nts 101-1011 and Glycoprotein = nts 1361-4768; RNA S-Silencing Suppressor = nts 91-1499 and Nucleoprotein = nts 2499-3277.
^{d}Region larger than 100 nts that was (1) identified from previous analysis of only TSWV sequences, (2) contains at least one 25 nucleotide fragment showing high similarity (greater than 92%) to a corresponding sequence in another tospovirus.
^{e}“>88% conserved” means that >88% of the nucleotide positions in EVERY 25 nucleotide fragment within this region are conserved in at least 95% of the sequences examined.
^{f}“100% conserved” means that 100% of the nucleotide positions in EVERY 25 nucleotide fragment within this region are conserved in at least 95% of the sequences examined.
^{g}All position numbers correspond to the nucleotide position along the consensus sequence derived from the alignment of TSWV genomic RNA L, M, or S, respectively.
^{h}No sequence corresponding to this area of the genomic RNA was available in GenBank.

### Example 2

### Transgene Expression of the Hairpins in Transgenic Events (TO)

With the exception of the C construct, multiple events of each construct (8-13 TO plants per construct) were used for evaluation. All eight of the TO events of the D transformants tested positive for the transgene and expressed the hairpins (Table 3). The other four transformation events (A, B, E, and F) resulted in 70%-90% transformation efficiency with 60%-90% of the plants testing positive for expression of the transgenes. The single C event tested positive for transgene presence and expression. However, it was discontinued from further testing due to its late entry into the transformation and screening pipeline.

Table 3. Percent of tomato plants that tested positive by PCR for transgene presence (DNA) and expression (RNA) in transformants (TO events) that harbor one of six different, multigenic hairpin constructs (A-F) designed to target all viral genes for degradation of multiple orthotospovirus species. Numbers in ( ) indicate the number of plants that survived after transplanting and evaluated for this analysis; *=only one event received.

| T0 events | +DNA (%) | +RNA (%) |
|---|---|---|
| A (10) | 90 | 90 |
| B (12) | 75 | 67 |
| C (1)* | 100 | 100 |
| D (8) | 100 | 100 |
| E (12) | 83 | 67 |
| F (10) | 70 | 60 |

### Example 3

### Multi-Generational Transgenic Tomato Lines Show Resistance to TSWV Delivered by Viruliferous Thrips

Thrips inoculation of transgenic events (TO) of three of the five construct designs (A, D and E) resulted in significantly lower (P<0.05) infection incidence (0% plants that tested positive to TSWV) compared to the shGFP negative control (100%) (Table 4), providing evidence to support resistance to TSWV (TSWV-HI isolate). With subsequent generations (T1 and T2), the transgenic progeny exhibited varied degrees of resistance to TSWV. Some lines with constructs B, E and F exhibited robust and sustained resistance (T1 and T2) to the virus as compared to the non-transgenic and/or shGFP controls (P<0.05), with E and F indicating an apparent homozygosity of the trait (0 plants testing positive for TSWV) by T2. Select lines with construct A expressed virus resistance at T2. With the entry of a California isolate of RB-TSWV into the test panel, it was determined that all four transgenes (A, B, E and F) expressed in T3 generation plants resulted in significant resistance (0% or 17% infected plants, P<0.05) compared to the shGFP transgenic control, non-transgenic control (‘Moneymaker’), and the Sw-5-containing non-transgenic control (‘Mountain Merit’). Although the D transgene construct appeared to be effective against TSWV infection at TO, progeny lines of this construct selected for testing in subsequent generations were as susceptible to the virus as the negative controls. As such, the D lines were not selected for further screening with the RB-TSWV isolate.

Table 4. Transgenic resistance to tomato spotted wilt virus (TSWV) and resistance-breaking (RB) TSWV inoculated by Frankliniella occidentalis females to leaf disks” sampled from tomato plants expressing one of five different, multigenic hairpin constructs (A-F) designed to target all viral genes for degradation.

| | | | Transgenic parent line or commercial variety | | | | | | | |
|---|---|---|---|---|---|---|---|---|---|---|
| | | | | | | | | | | MMT |
| TSWV | | | | | | | | | MM | (Sw-5) |
| isolate | Generation^{b} | Unite | A | B | D | E | F | shGFP | Control | Control |
| Hawaii | T0 | Leaf | 0/20 | 1/8 | 0/8 | 0/20 | 1/20 | 9/12 | nt^{d} | nt |
| (MT2) | | disk | (0%) | (13%) | (0%) | (0%) | (5%) | (75%) | | |
| | T0 | Plant | 0/5* | 1/2 | 0/2* | 0/5* | 1/5 | 3/3 | nt | nt |
| | | | (0%) | (50%) | (0%) | (0%) | (20%) | (100%) | | |
| | T1 | Leaf | 8/36 | 0/9 | 6/18 | 8/42 | 1/21 | nt | 10/15 | nt |
| | | disk | (22%) | (0%) | (33%) | (19%) | (5%) | | (67%) | |
| | T1 | Plant | 7/12 | 0/3* | 4/6 | 5/14* | 1/7* | nt | 5/5 | nt |
| | | | (58%) | (0%) | (67%) | (36%) | (14%) | | (100%) | |
| | T2 | Leaf | 1/27 | 2/27 | 6/9 | 0/39 | 0/27 | 8/9 | 12/15 | nt |
| | | disk | (4%) | (7%) | (67%) | (0%) | (0%) | (89%) | (80%) | |
| | T2 | Plant | 1/9* | 1/9* | 3/3 | 0/13* | 0/9* | 3/3 | 5/5 | nt |
| | | | (11%) | (11%) | (100%) | (0%) | (0%) | (100%) | (100%) | |
| California | T3 | Leaf | 0/18 | 0/18 | nt | 0/18 | 1/18 | 14/18 | 12/18 | 11/18 |
| (RB-TSWV) | | disk | (0%) | (0%) | | (0%) | (6%) | (78%) | (67%) | (61%) |
| | T3 | Plant | 0/6* | 0/6* | nt | 0/6* | 1/6* | 6/6 | 5/6 | 6/6 |
| | | | (0%) | (0%) | | (0%) | (17%) | (100%) | (83%) | (100%) |

^{a}Cohorts of viruliferous females were allowed a 48-hour (T0-T2) or 72-hour (T3) inoculation access period on three leaf disks per plant for multiple plants per transgenic line or commercial variety; thrips were removed and leaf discs were incubated for four days to allow for virus accumulation. TSWV was detected by ELISA; shGFP = negative transgenic control expressing short-hairpins (sh) of GFP in cv. ‘Moneymaker’ background; MM = ‘Moneymaker’ non-transgenic control; MMT = ‘Mountain Merit’ non-transgenic control carries the tospovirus resistance gene Sw-5.
^{b}Assays were performed with transgenic events (T0) and three subsequent generations (T1-T3) of each transgenic line.
^{c}Unit for assessment of virus infection incidence, i.e., number of ELISA-positive leaf disks/total disks tested and number of ELISA-positive plants/total plants tested per line or variety; a plant was considered infected if at least one of the three leaf disks tested positive for virus. The value in ( ) is percent infection for that unit.
^{d}not tested.
*= statistically-different (two-sided p-value < 0.05) from hairpin negative control tomato (shGFP) and non-transgenic controls [MM or MMT] determined by a Fisher's Exact Test of Independence on rxc contingency table [infection status × line/variety] followed by pairwise exact test between antiviral hairpin lines and shGFP or non-transgenic variety (P < 0.05).

### Example 4

Transgenic Resistance of Promising Lineages Confirmed in Whole Plant Experiments by Mechanical Rub Inoculation with Virus

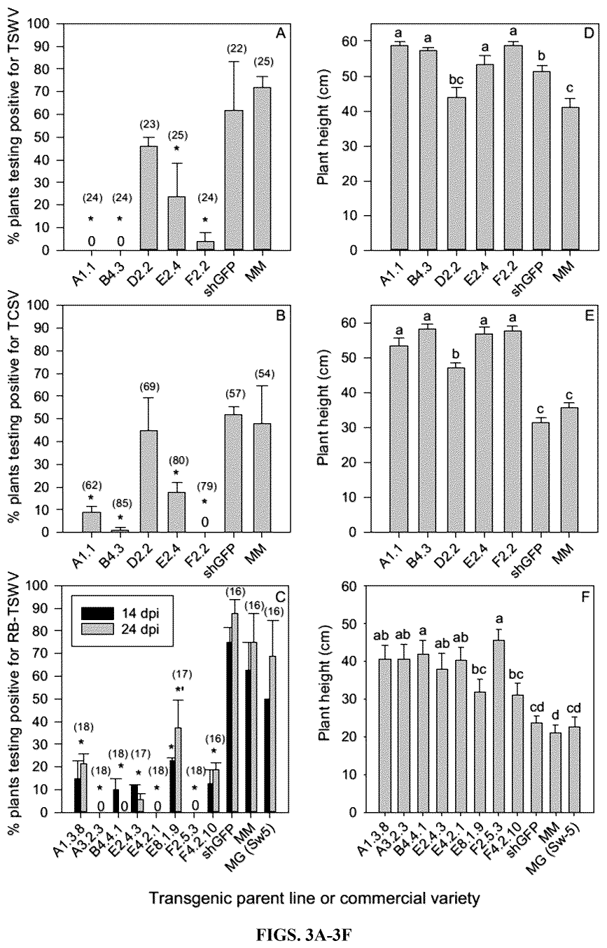

T2 generation plants (e.g., progeny of T1 parent lines) carrying the A, B, E or F transgenes had low virus infection rates (incidence of infection) when challenged with TSWV-HI and TCSV-FL (FIGS. 3A and 3B, P<0.05) compared to the T2 plants of the shGFP and non-transgenic susceptible controls (‘Moneymaker’). Low infection rates translated into 11% and 79% taller plants (on average for TSWV-HI and TCSV-FL, respectively) compared to shGFP, and 39% and 59% taller plants, respectively, compared to ‘Moneymaker’ susceptible controls (FIGS. 3D and 3E, P<0.05). T3 plants of multiple A, E, and F transgenic lines and one B line mechanically inoculated with the RB-TSWV-CA isolate exhibited significant reductions in infection incidence (P<0.05) at both sampling time points (14 and 24 dpi) compared to shGFP transgenics, ‘Moneymaker’ and ‘Mountain Glory’ (Sw-5) susceptible controls (FIG. 3C). While typical symptoms of infection were observed on the control plants, none of the hairpin transgenic test plants that tested negative for TSWV expressed visual leaf or stem symptoms typical of TSWV. Plants of both A lines, one E, F and the B line were significantly taller on average (80%-100%) than the three susceptible controls (FIG. 3F) by 24 dpi. While only one trial was performed with TSWV-NC (non-RB isolate) on the same T2 parent lines used for the RB-TSWV-CA experiments (see FIG. 3C for line IDs), it was determined that all but two of the lines (E.8.1, F4.2.1) were resistant to the NC isolate (0% infected plants, no apparent symptomology) compared to 100% infection rate of the shGFP and ‘Moneymaker’ controls (TSWV symptoms observed), and 0% infection coupled with no apparent symptoms of the Sw-5 cultivar ‘Mountain Glory’ (data not shown). In total, lineages with A, B, E and F constructs showed resistance to TSWV, TCSV and RB-TSWV.

### Example 6

### Flowers and Fruit Express Antiviral Hairpin Transgenes

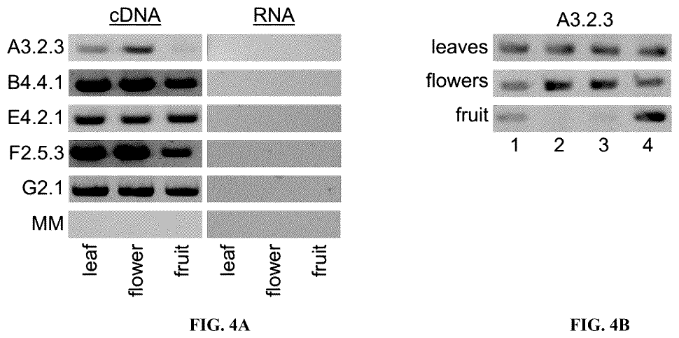

Transgene expression was detected in leaf, flower, and fruit tissues of T3 plants by endpoint RT-PCR using primers for the hairpin GUS-linker (FIG. 4). Amplicons of the expected 639-bp size were visible for all transgenic lines, compared to no product for ‘Moneymaker’ negative controls. Detection of transgene expression in line A3.2.3 was variable across individual fruits, however there was consistent expression in leaves and flowers of this line. The absence of product from DNase-treated RNA from transgenic tissues prior to cDNA synthesis confirmed that positive evidence of amplicons indicated RNA and not residual DNA template (e.g., expression).

Table 5. Primers used for cloning, sequencing, and transgene detection of multigenic hairpin constructs (A to F) designed to target tomato spotted wilt virus (TSWV) and related orthotospoviruses.

| Name | Sequence (5′-3′) | Purpose |
|---|---|---|
| TSWVA-F | GCCACCGGTCATGTGTATATCCC | Amplification of TSWV-A hairpin |
| | (SEQ ID NO: 24) | arm |
| TSWVA-R | GCCGTCGACAAGTTGAATTCCCC | Amplification of TSWV-A hairpin |
| | (SEQ ID NO: 25) | arm |
| TSWVB-F | GCCACCGGTCATCTTCCTTTGG | Amplification of TSWV-B hairpin |
| | (SEQ ID NO: 26) | arm |
| TSWVB-R | GCCGTCGACATACTTAGTTCCAGC | Amplification of TSWV-B hairpin |
| | (SEQ ID NO: 27) | arm |
| TSWVC-F | CCGGTATATTCCGACAGAATCCGC | Amplification of TSWV-C hairpin |
| | (SEQ ID NO: 28) | arm |
| TSWVC-R | CTCCCCTGGCAAAGTCTATCTTCC | Amplification of TSWV-C hairpin |
| | (SEQ ID NO: 29) | arm |
| TSWVD-F | GCCACCGGTCCTACAAAAGG (SEQ | Amplification of TSWV-D hairpin |
| | ID NO: 30) | arm |
| TSWVD-R | GCCGTCGACTGCTCTATAAAATAA | Amplification of TSWV-D hairpin |
| | AGCATC (SEQ ID NO: 31) | arm |
| TSWVE-F | CGGTGCCTGTTTTTTAACCCC (SEQ | Amplification of TSWV-E hairpin |
| | ID NO: 32) | arm |
| TSWVE-R | GCCGTCGACAGAATGACTTGC | Amplification of TSWV-E hairpin |
| | (SEQ ID NO: 33) | arm |
| TSWVF-F | CGGTAAAATGTCACCGGGTCTG | Amplification of TSWV-F hairpin |
| | (SEQ ID NO: 34) | arm |
| TSWVF-R | ATGAGGCCGTCGACTTATTCTGG | Amplification of TSWV-F hairpin |
| | (SEQ ID NO: 35) | arm |
| JEO- | CACGCAAGTCCGCATCTTCA (SEQ | Sequencing of constructs in |
| GUSF1 | ID NO: 36) | pANDA35HK |
| GUSR2 | GTATCAGTGTGCATGGCTGG (SEQ | Sequencing of constructs in |
| | ID NO: 37) | PANDA35HK |
| GUS-F | CATGAAGATGCGGACTTGCG (SEQ | Amplification of GUS linker |
| | ID NO: 38) | |
| GUS-R | ATCCACGCCGTATTCGG (SEQ ID | Amplification of GUS linker |
| | NO: 39) | |

### Example 7

Transgenic Tomato Fruits are Protected from Non-RB and RB TSWV Infection

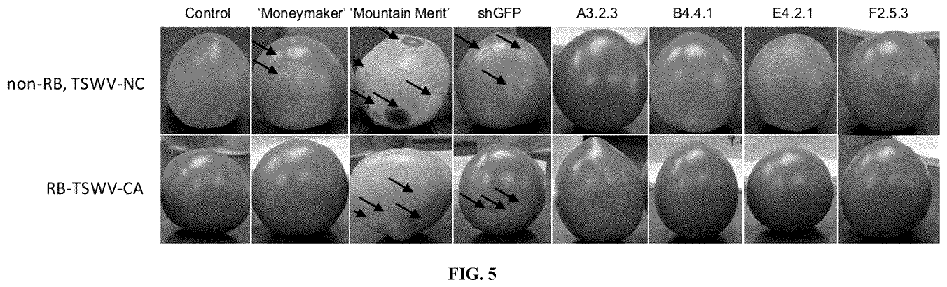

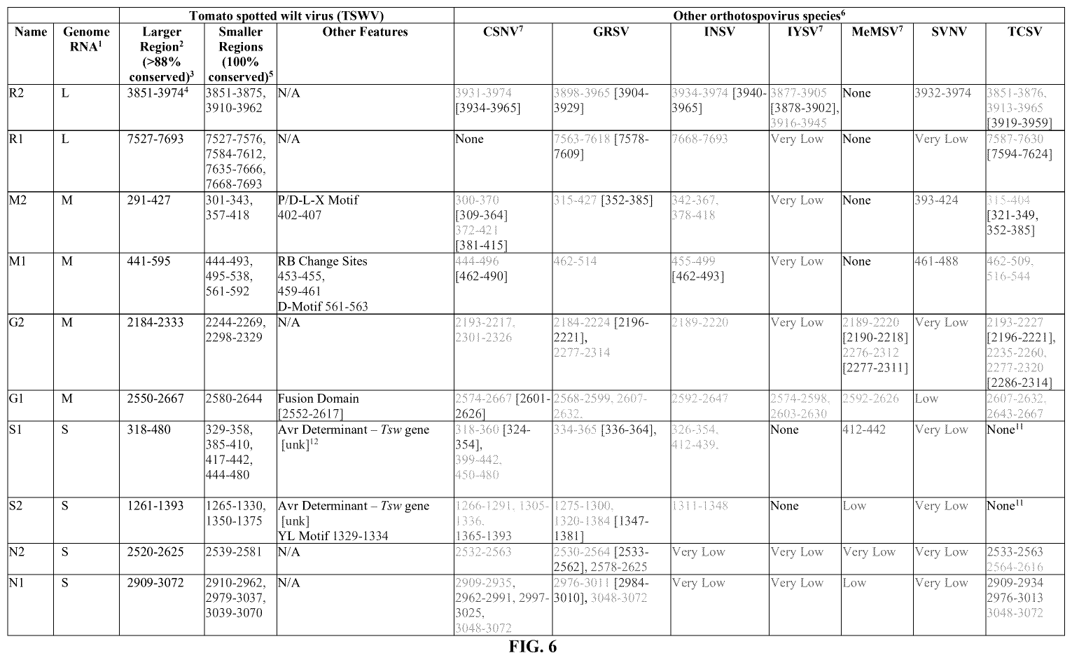

Visible TSWV symptoms and virus infection status, as measured by DAS-ELISA, were used to determine transgenic fruit resistance to TSWV. Thrips feeding scars were apparent on the fruit in the thrips-feeding arenas regardless of treatment (all controls and transgenic hairpin test lines). Fruit from the transgenic control (shGFP in ‘Moneymaker’ background) reliably developed symptoms typical of TSWV infection (˜1-cm² chlorotic, circular clearances) roughly two weeks post exposure to viruliferous adults infected with non-RB or RB-TSWV (FIG. 5). On the contrary, the phenotypic response of the commercial cultivar controls varied by strain. ‘Moneymaker’ (non-resistant) symptomatology mirrored that of the shGFP fruit in the non-RB TSWV screen. However, when challenged with RB-TSWV, positive-testing fruit of this cultivar expressed no apparent symptoms. Symptomatic ‘Mountain Merit’ (Sw-5-containing) fruit developed necrotic rings in the chlorotic clearances when infected with non-RB-TSWV, in contrast to no necrotic rings when infected with RB-TSWV (FIG. 5). Although fruit symptom expression differed among the three control groups, pericarp tissues consistently tested positive for TSWV regardless of the strain. For the non-RB-TSWV experiment, total infection incidence over all three independent trials was 81.2%, 87.5%, 64.3% for ‘Moneymaker’, ‘Mountain Merit’, and shGFP fruits, respectively (Table 6). For the RB-TSWV experiment, total infection incidence was 94.1%, 100%, 94.1% for ‘Moneymaker’, ‘Mountain Merit’, and shGFP respectively (Table 6). In contrast, none of the hairpin (antiviral) transgenic fruit (131 in total) from the four best-performing T3 plants developed symptoms nor tested positive for TSWV, regardless of TSWV strain (Table 6).

Table 6. Transgenic resistance to non-resistance breaking (non-RB, NC isolate) and resistance-breaking (RB, California isolate) TSWV inoculated by Frankliniella occidentalis on detached fruit from tomato plants expressing multigenic hairpin constructs (A, B, E, and F) a designed to target all viral genes for degradation.

| | | No virus | | MMT | | | | | |
|---|---|---|---|---|---|---|---|---|---|
| TSWV | | thrips | MM | control | | | | | |
| isolate | Trial^{b} | control | control | (Sw-5) | shGFP | A3.2.3 | B4.4.1 | E4.2.1 | F2.5.3 |
| non-RB- | 1 | 0/5^{c} | 2/5 | 3/5 | 1/4 | 0/5 | 0/5 | 0/5 | 0/5 |
| TSWV | 2 | 0/6 | 5/5 | 5/5 | 3/5 | 0/5 | 0/5 | 0/5 | 0/5 |
| (NC) | 3 | 0/6 | 6/6 | 6/6 | 5/5 | 0/6 | 0/6 | 0/5 | 0/6 |
| RB-TSWV | 1 | 0/7 | 4/5 | 5/5 | 5/5 | 0/5 | 0/5 | 0/5 | 0/5 |
| (CA) | 2 | 0/7 | 6/6 | 6/6 | 5/6 | 0/6 | 0/6 | 0/6 | 0/6 |
| | 3 | 0/7 | 6/6 | 6/6 | 6/6 | 0/6 | 0/6 | 0/6 | 0/6 |

^{a}T3 generation plants from T2 parents lines.
^{b}Biological repetitions of the experiment, i.e., independent trials.
^{c}Number of fruits testing positive for TSWV via DAS-ELISA/number of fruits inoculated per arena.
shGFP = short-hairpin transgenic control (GFP), MM = cv. ‘Moneymaker’ (non-transgenic control background of antiviral constructs, no Sw-5 R gene); ‘MMT’ = cv. ‘Mountain Merit’ (non-transgenic, Sw-5 R gene containing tomato).

Due to the propensity for rapid genetic change of segmented viruses by reassortment of genome segments, the approach simultaneously targeted all three genome segments and the five opening reading frames. By stacking the RNAi targets to include the complete genome, these hairpin constructs may provide long lasting and durable protection and, as the results indicate thus far, broad spectrum resistance to tomato-infecting tospoviruses. The RNA targets identified in the sequence analysis pipeline all match coding regions of the TSWV genome that are transcribed into viral mRNA. This is an important consideration for the RNAi approach because orthotospovirus genomic RNAs are coated in nucleocapsid protein and not the target of the RNAi pathway. By specifically including sequences in the hairpin that are highly conserved and map to known functional domains of proteins, the likelihood that they will be targeted by the plant RNAi machinery and that mutations that develop in those coding sequences will have a fitness cost for the virus is increased.

The concatenated dsRNA hairpins developed provided effective control for TSWV isolates from different U.S. tomato production regions including RB-TSWV from tomato production areas in CA and an isolate of TSWV recently obtained from fresh market tomatoes in NC. Resistance extended to other tospoviruses as demonstrated with TCSV, a closely related virus to TSWV. Regardless of how the virus was delivered in resistance screens, e.g., thrips-inoculation or mechanical leaf-rub inoculation, the hairpin-mediated, transgenic resistance was robustly demonstrated in the most promising lineages. As a potential added benefit to growers, the present study also documented fruit protection against TSWV delivered by viruliferous adult thrips, and evidence indicated that the hairpin transgenes are expressed in the fruit pericarp. The Sw-5 gene does not provide protection against flower or fruit infection, and occurrence of high thrips and TSWV pressures, even with the use of Sw-5 tomatoes, the crop can experience significant yield loss. The lack of fruit resistance by the Sw-5 gene has also complicated the identification of RB-TSWV isolates because the infection of fruit by non-RB-TSWV can result in typical TSWV symptoms on these tissues.

Table 7. References for conserved protein domains previously identified within the TSWV genome.

| Genomic | | | | |
|---|---|---|---|---|
| RNA | Genomic Region¹ | Location² | Feature | Reference |
| L | RdRp | 4111-4161 | RdRp Motif A | Peng et al. 2014 [PLOS One] |
| L | RdRp | 4389-4458 | RdRp Motif B | Peng et al. 2014 [PLOS One] |
| L | RdRp | 4519-4551 | RdRp Motif C | Peng et al. 2014 [PLOS One] |
| L | RdRp | 4630-4683 | RdRp Motif D | Peng et al. 2014 [PLOS One] |
| L | RdRp | 4684-4716 | RdRp Motif E | Peng et al. 2014 [PLOS One] |
| M | Movement Protein | 402-407 | P/D-L-X-motif | Silva et al. 2001 [Arch. Of |
| | (NSm) | | | Virol.] |
| M | Movement Protein | 453-455 | Resistance Breakdown AA | Peiro et al. 2014 [MPP] |
| | (NSm) | | change C118Y | |
| M | Movement Protein | 459-461 | Resistance Breakdown AA | Peiro et al. 2014 [MPP] |
| | (NSm) | | change T118N | |
| M | Movement Protein | 561-563 | D-motif | Silva et al. 2001 [Arch. Of |
| | (NSm) | | | Virol.] |
| M | Movement Protein | 684-707 | PLA2-motif | Silva et al. 2001 [Arch. Of |
| | (NSm) | | | Virol.] |
| M | Movement Protein | 726-728 | G-residue | Silva et al. 2001 [Arch. Of |
| | (NSm) | | | Virol.] |
| M | Glycoprotein (GC) | 1490-1567 | Transmembrane domain | Garry and Garry 2004 [BioMed |
| | | | | Central] |
| M | Glycoprotein (GC) | 2552-2617 | Fusion peptide | Garry and Garry 2004 [BioMed |
| | | | | Central] |
| S | Silencing Suppressor | 633-641 | GKV Motif | Zhai et al. 2014 [Arch. Of |
| | (NSs) | | | Virol.] |
| S | Silencing Suppressor | 1329-1334 | YL Motif | Zhai et al. 2014 [Arch. Of |
| | (NSs) | | | Virol.] |
| S | Silencing Suppressor | Unknown | Avr Determinant-Tsw R | De Ronde et al. 2013 [MPP] |
| | (NSs) | | gene (pepper) | |

¹Coding Sequences correspond to the following positions within each respective RNA: RNA L-nts 35-8690 (RdRp); RNA M-nts 101-1011 (Movement Protein), nts 1361-4768 (Glycoprotein); RNA S-nts 91-1499 (Silencing Suppressor), nts. 2499-3277 (Nucleoprotein).
²Location relative to the generated consensus sequence used in conserved region analysis.

1. An RNA hairpin comprising a stem region comprising one or more polynucleotide sequences, wherein each of the one or more polynucleotide sequences individually has at least 70% identity to a sequence from a target virus.

2. The RNA hairpin of claim 1, wherein the sequence from the target virus is from a conserved region of the target virus genome.

3. The RNA hairpin of claim 2, wherein the conserved region includes regions of at least 25 nucleotides in which at least 85% of the nucleotide positions are conserved.

4-5. (canceled)

6. The RNA hairpin of claim 1, wherein the sequence from the target virus is from a protein-encoding region of the target virus.

7. The RNA hairpin of claim 6, wherein the sequence from the target virus is in a domain which controls protein function.

8. The RNA hairpin of claim 1, wherein the sequence from the target virus is 100 to 200 nucleotides in length.

9. The RNA hairpin of claim 1, wherein:
each of the one or more polynucleotide sequences individually has at least 70% identity to a sequence from a different gene of the target virus;
the one or more polynucleotide sequences include in total at least one sequence with at least 70% identity to a sequence from each gene of the target virus; or
the one or more polynucleotide sequences include in total at least one sequence with at least 70% identity to a sequence from each open reading frame of the target virus.

10. The RNA hairpin of any one of claims 1-9, wherein the one or more polynucleotide sequences include in total at least one sequence with at least 70% identity to a sequence from each open reading frame of the target virus.

11. The RNA hairpin of any one of claims 1-10, wherein the one or more polynucleotide sequences include in total at least one sequence with at least 70% identity to a sequence from each open reading frame of the target virus.

12. The RNA hairpin of claim 1, wherein the one or more polynucleotide sequences are selected from SEQ ID NOs: 1-10.

13. The RNA hairpin of claim 1, wherein the RNA hairpin further comprises a loop region having a single stranded linker sequence.

14. The RNA hairpin of claim 13, wherein the single stranded linker sequence is fragment from the glucuronidase gene of E. coli.

15. The RNA hairpin of claim 1, wherein the target virus is one or more orthotospoviruses.

16. The RNA hairpin of claim 15, wherein the one or more orthotospoviruses is tomato spotted wilt virus (TSWV), tomato chlorotic spot virus (TCSV), groundnut ringspot virus (GRSV), impatiens necrotic spot virus (INSV), iris yellow spot virus (IYSV), melon severe mosaic virus (MeSMV), chrysanthemum stem necrosis virus (CSNV), soybean vein necrosis virus (SVNV), or a combination thereof.

17-18. (canceled)

19. The RNA hairpin of claim 1, comprising a nucleotide sequence of any of SEQ ID NOs: 11-16 and 18-23.

20-22. (canceled)

23. A method of increasing virus resistance or preventing viral infection in a plant comprising providing an RNA hairpin of claim 1, or a nucleic acid encoding thereof, to the plant, or a plant cell, seed, fruit, plant part, or propagation material of the plant.

24-26. (canceled)

27. The method of claim 23, wherein the plant is a food or ornamental crop.

28. The method of claim 23, wherein the plant is a tomato.

29. The method of claim 23, further comprising providing another viral resistance treatment to the plant.

30. A plant, plant cell, seed, fruit, plant part, or propagation material of the plant comprising an RNA hairpin of claim 1, or a nucleic acid encoding thereof.

31. The plant, plant cell, seed, fruit, plant part, or propagation material of the plant of claim 30, wherein the plant, plant cell, seed, fruit, plant part, or propagation material of the plant has increased viral resistance as compared to a plant, plant cell, seed, fruit, plant part, or propagation material of the plant not comprising the RNA hairpin or a nucleic acid encoding thereof.

32. (canceled)

33. A method of generating an antiviral hairpin construct comprising:
identifying conserved region of a target virus genome;
selecting sequences from the conserved regions which are in protein-encoding domains; and
compiling selected sequences for each gene or open reading frame of the target virus into a single concatenated sequence; and
generating the antiviral hairpin construct with the following regions i) a first region comprising compiled sequences, ii) a second region comprising a linker sequence configured to form a loop, and iii) a third region substantially complementary to the compiled sequences configured to form a double stranded stem with the first region.

34-37. (canceled)


SEQUENCE LISTING (39 sequences)

SEQ ID NO: 1 - DNA, 167 bp, Synthetic construct
  source 1..167: mol_type = other DNA
aaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaactt

SEQ ID NO: 2 - DNA, 124 bp, Synthetic construct
  source 1..124: mol_type = other DNA
aaaatgtcaccgggtctgtagatttcctagtttctgtttttgagaaaatgcagagaaccaaaacagacagagaaatatacttgatgagcatgaaagtgaaaatgatgctttattttatagagca

SEQ ID NO: 3 - DNA, 155 bp, Synthetic construct
  source 1..155: mol_type = other DNA
gtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattct

SEQ ID NO: 4 - DNA, 136 bp, Synthetic construct
  source 1..136: mol_type = other DNA
catcttcctttggaacctatgaatctgattctatcacagaatcagagggttatgatctttctgcaagaatgatagtagatacaaaccatcatatctcaaactggaaaaatgatctttttgttggcaacgggaagca

SEQ ID NO: 5 - DNA, 118 bp, Synthetic construct
  source 1..118: mol_type = other DNA
atattccgacagaatccgcatgtagcaccttcattaattgcaaaacaccaagcttcttcacatccccaataagaagttggtgttatacaaaaatcttggaaacctgttaaagcttgat

SEQ ID NO: 6 - DNA, 150 bp, Synthetic construct
  source 1..150: mol_type = other DNA
attgattttttccctattgctgcacaatcccaagacatgtcatctccttctagagttttcttagtaaaaataggcactccatcatgagtcaattgtgggtgaccaaacattttcacaggatcattcaagtttgcaatatttccagaataa

SEQ ID NO: 7 - DNA, 163 bp, Synthetic construct
  source 1..163: mol_type = other DNA
catgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatcta

SEQ ID NO: 8 - DNA, 133 bp, Synthetic construct
  source 1..133: mol_type = other DNA
cctacaaaaggaaatatttcctttcaaaaacacttgaatgtcttccatctaacacacaaactatgtcttatttagacagcatccaaattccttcctggaagatagactttgccaggggagaaattaaaatttc

SEQ ID NO: 9 - DNA, 164 bp, Synthetic construct
  source 1..164: mol_type = other DNA
ggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcacttt

SEQ ID NO: 10 - DNA, 105 bp, Synthetic construct
  source 1..105: mol_type = other DNA
tgcctgttttttaaccccaaacatttcatagaacttgttaagagtttcactgtaatgttccatagcaacacttcctttagcattaggattgctggaactaagtat

SEQ ID NO: 11 - DNA, 824 bp, Synthetic construct
  source 1..824: mol_type = other DNA
ggccgtcaaggccaccggtcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctactagtatattccgacagaatccgcatgtagcaccttcattaattgcaaaacaccaagcttcttcacatccccaataagaagttggtgttatacaaaaatcttggaaacctgttaaagcttgatcgatgtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctcctaggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttcatatgaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttgtcgacggcctcatgggcc

SEQ ID NO: 12 - DNA, 704 bp, Synthetic construct
  source 1..704: mol_type = other DNA
ggccgtcaaggccaccggtcatcttcctttggaacctatgaatctgattctatcacagaatcagagggttatgatctttctgcaagaatgatagtagatacaaaccatcatatctcaaactggaaaaatgatctttttgttggcaacgggaagcactagtcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctatcgataaaatgtcaccgggtctgtagatttcctagtttctgtttttgagaaaatgcagagaaccaaaacagacagagaaatatacttgatgagcatgaaagtgaaaatgatgctttattttatagagcacctaggatattccgacagaatccgcatgtagcaccttcattaattgcaaaacaccaagcttcttcacatccccaataagaagttggtgttatacaaaaatcttggaaacctgttaaagcttgatcatatgcctgttttttaaccccaaacatttcatagaacttgttaagagtttcactgtaatgttccatagcaacacttcctttagcattaggattgctggaactaagtatgtcgacggcctcatgggcc

SEQ ID NO: 13 - DNA, 779 bp, Synthetic construct
  source 1..779: mol_type = other DNA
ggccgtcaaggccaccggtatattccgacagaatccgcatgtagcaccttcattaattgcaaaacaccaagcttcttcacatccccaataagaagttggtgttatacaaaaatcttggaaacctgttaaagcttgatactagtcatcttcctttggaacctatgaatctgattctatcacagaatcagagggttatgatctttctgcaagaatgatagtagatacaaaccatcatatctcaaactggaaaaatgatctttttgttggcaacgggaagcatcgatggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttcctaggaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttcatatgcctacaaaaggaaatatttcctttcaaaaacacttgaatgtcttccatctaacacacaaactatgtcttatttagacagcatccaaattccttcctggaagatagactttgccaggggagaaattaaaatttcgtcgacggcctcatgggcc

SEQ ID NO: 14 - DNA, 785 bp, Synthetic construct
  source 1..785: mol_type = other DNA
ggccgtcaaggccaccggtcctacaaaaggaaatatttcctttcaaaaacacttgaatgtcttccatctaacacacaaactatgtcttatttagacagcatccaaattccttcctggaagatagactttgccaggggagaaattaaaatttcactagtggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttatcgattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataacctaggtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctcatatgaaaatgtcaccgggtctgtagatttcctagtttctgtttttgagaaaatgcagagaaccaaaacagacagagaaatatacttgatgagcatgaaagtgaaaatgatgctttattttatagagcagtcgacggcctcatgggcc

SEQ ID NO: 15 - DNA, 800 bp, Synthetic construct
  source 1..800: mol_type = other DNA
ggccgtcaaggccaccggtgcctgttttttaaccccaaacatttcatagaacttgttaagagtttcactgtaatgttccatagcaacacttcctttagcattaggattgctggaactaagtatactagtaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttatcgatcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctacctaggattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataacatatgtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctgtcgacggcctcatgggcc

SEQ ID NO: 16 - DNA, 706 bp, Synthetic construct
  source 1..706: mol_type = other DNA
ggccgtcaaggccaccggtaaaatgtcaccgggtctgtagatttcctagtttctgtttttgagaaaatgcagagaaccaaaacagacagagaaatatacttgatgagcatgaaagtgaaaatgatgctttattttatagagcactagttgcctgttttttaaccccaaacatttcatagaacttgttaagagtttcactgtaatgttccatagcaacacttcctttagcattaggattgctggaactaagtatcgatcatcttcctttggaacctatgaatctgattctatcacagaatcagagggttatgatctttctgcaagaatgatagtagatacaaaccatcatatctcaaactggaaaaatgatctttttgttggcaacgggaagcacctaggcctacaaaaggaaatatttcctttcaaaaacacttgaatgtcttccatctaacacacaaactatgtcttatttagacagcatccaaattccttcctggaagatagactttgccaggggagaaattaaaatttcatatgattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataagtcgacggcctcatgggcc

SEQ ID NO: 17 - DNA, 929 bp, Synthetic construct
  source 1..929: mol_type = other DNA
atctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcg

SEQ ID NO: 18 - DNA, 2736 bp, Synthetic construct
  source 1..2736: mol_type = other DNA
gcccatgaggccgtcgacaagttgaattcccctatctctatgtgcttttttgccattagttcaacagcatgcattccagtggctctatagcttttaaatcctgatgcatcatttatcagagaatatttaagcatgagcagtatttctttgaaaccttctaaatctctagtatactgttttgctttcatatgaaagtgattttacttttggtaaaattaccataaagaaaacttcagacaggattggagccactgacatgaccttcagaaggcttgatagcttgatcagggtcaggcttgttgaggaaactgggaattctgagaatctcaatactatcaaatctaagattgcttcctaggagaatgacttgcttttcagatggcatgttgggatcaaccagggcaaccacaagtttccctgtagggtttggtatagtggggcagacccatatcacaatcctggaaatcatcatgtattgttttctgctgtcccaagttggacagatcttgatgacatcgatcaagctttaacaggtttccaagatttttgtataacaccaacttcttattggggatgtgaagaagcttggtgttttgcaattaatgaaggtgctacatgcggattctgtcggaatatactagtagatccaatagagtggagctgagcaggagacaaaaccttcaaatgaccttgatgtttcactccgttagcattgactgtatttgagcaaacagatagtgccagaacagagtcatcaatattgatgctaaaatcaatatcatcaaaaatagggatatacacatgaccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctactagtatattccgacagaatccgcatgtagcaccttcattaattgcaaaacaccaagcttcttcacatccccaataagaagttggtgttatacaaaaatcttggaaacctgttaaagcttgatcgatgtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctcctaggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttcatatgaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttgtcgacggcctcatgggc

SEQ ID NO: 19 - DNA, 2496 bp, Synthetic construct
  source 1..2496: mol_type = other DNA
gcccatgaggccgtcgacatacttagttccagcaatcctaatgctaaaggaagtgttgctatggaacattacagtgaaactcttaacaagttctatgaaatgtttggggttaaaaaacaggcatatgatcaagctttaacaggtttccaagatttttgtataacaccaacttcttattggggatgtgaagaagcttggtgttttgcaattaatgaaggtgctacatgcggattctgtcggaatatcctaggtgctctataaaataaagcatcattttcactttcatgctcatcaagtatatttctctgtctgttttggttctctgcattttctcaaaaacagaaactaggaaatctacagacccggtgacattttatcgatagatccaatagagtggagctgagcaggagacaaaaccttcaaatgaccttgatgtttcactccgttagcattgactgtatttgagcaaacagatagtgccagaacagagtcatcaatattgatgctaaaatcaatatcatcaaaaatagggatatacacatgactagtgcttcccgttgccaacaaaaagatcatttttccagtttgagatatgatggtttgtatctactatcattcttgcagaaagatcataaccctctgattctgtgatagaatcagattcataggttccaaaggaagatgaccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtcatcttcctttggaacctatgaatctgattctatcacagaatcagagggttatgatctttctgcaagaatgatagtagatacaaaccatcatatctcaaactggaaaaatgatctttttgttggcaacgggaagcactagtcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctatcgataaaatgtcaccgggtctgtagatttcctagtttctgtttttgagaaaatgcagagaaccaaaacagacagagaaatatacttgatgagcatgaaagtgaaaatgatgctttattttatagagcacctaggatattccgacagaatccgcatgtagcaccttcattaattgcaaaacaccaagcttcttcacatccccaataagaagttggtgttatacaaaaatcttggaaacctgttaaagcttgatcatatgcctgttttttaaccccaaacatttcatagaacttgttaagagtttcactgtaatgttccatagcaacacttcctttagcattaggattgctggaactaagtatgtcgacggcctcatgggc

SEQ ID NO: 20 - DNA, 2648 bp, Synthetic construct
  source 1..2648: mol_type = other DNA
ggcccatgaggccgtcgacgaaattttaatttctcccctggcaaagtctatcttccaggaaggaatttggatgctgtctaaataagacatagtttgtgtgttagatggaagacattcaagtgtttttgaaaggaaatatttccttttgtaggcatatgaagttgaattcccctatctctatgtgcttttttgccattagttcaacagcatgcattccagtggctctatagcttttaaatcctgatgcatcatttatcagagaatatttaagcatgagcagtatttctttgaaaccttctaaatctctagtatactgttttgctttcctaggaaagtgattttacttttggtaaaattaccataaagaaaacttcagacaggattggagccactgacatgaccttcagaaggcttgatagcttgatcagggtcaggcttgttgaggaaactgggaattctgagaatctcaatactatcaaatctaagattgcttccatcgatgcttcccgttgccaacaaaaagatcatttttccagtttgagatatgatggtttgtatctactatcattcttgcagaaagatcataaccctctgattctgtgatagaatcagattcataggttccaaaggaagatgactagtatcaagctttaacaggtttccaagatttttgtataacaccaacttcttattggggatgtgaagaagcttggtgttttgcaattaatgaaggtgctacatgcggattctgtcggaatataccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtatattccgacagaatccgcatgtagcaccttcattaattgcaaaacaccaagcttcttcacatccccaataagaagttggtgttatacaaaaatcttggaaacctgttaaagcttgatactagtcatcttcctttggaacctatgaatctgattctatcacagaatcagagggttatgatctttctgcaagaatgatagtagatacaaaccatcatatctcaaactggaaaaatgatctttttgttggcaacgggaagcatcgatggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttcctaggaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttcatatgcctacaaaaggaaatatttcctttcaaaaacacttgaatgtcttccatctaacacacaaactatgtcttatttagacagcatccaaattccttcctggaagatagactttgccaggggagaaattaaaatttcgtcgacggcctcatgggcc

SEQ ID NO: 21 - DNA, 2660 bp, Synthetic construct
  source 1..2660: mol_type = other DNA
ggcccatgaggccgtcgactgctctataaaataaagcatcattttcactttcatgctcatcaagtatatttctctgtctgttttggttctctgcattttctcaaaaacagaaactaggaaatctacagacccggtgacattttcatatgagaatgacttgcttttcagatggcatgttgggatcaaccagggcaaccacaagtttccctgtagggtttggtatagtggggcagacccatatcacaatcctggaaatcatcatgtattgttttctgctgtcccaagttggacagatcttgatgacctaggttattctggaaatattgcaaacttgaatgatcctgtgaaaatgtttggtcacccacaattgactcatgatggagtgcctatttttactaagaaaactctagaaggagatgacatgtcttgggattgtgcagcaatagggaaaaaatcaatcgataaagtgattttacttttggtaaaattaccataaagaaaacttcagacaggattggagccactgacatgaccttcagaaggcttgatagcttgatcagggtcaggcttgttgaggaaactgggaattctgagaatctcaatactatcaaatctaagattgcttccactagtgaaattttaatttctcccctggcaaagtctatcttccaggaaggaatttggatgctgtctaaataagacatagtttgtgtgttagatggaagacattcaagtgtttttgaaaggaaatatttccttttgtaggaccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtcctacaaaaggaaatatttcctttcaaaaacacttgaatgtcttccatctaacacacaaactatgtcttatttagacagcatccaaattccttcctggaagatagactttgccaggggagaaattaaaatttcactagtggaagcaatcttagatttgatagtattgagattctcagaattcccagtttcctcaacaagcctgaccctgatcaagctatcaagccttctgaaggtcatgtcagtggctccaatcctgtctgaagttttctttatggtaattttaccaaaagtaaaatcactttatcgattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataacctaggtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctcatatgaaaatgtcaccgggtctgtagatttcctagtttctgtttttgagaaaatgcagagaaccaaaacagacagagaaatatacttgatgagcatgaaagtgaaaatgatgctttattttatagagcagtcgacggcctcatgggcc

SEQ ID NO: 22 - DNA, 2688 bp, Synthetic construct
  source 1..2688: mol_type = other DNA
gcccatgaggccgtcgacagaatgacttgcttttcagatggcatgttgggatcaaccagggcaaccacaagtttccctgtagggtttggtatagtggggcagacccatatcacaatcctggaaatcatcatgtattgttttctgctgtcccaagttggacagatcttgatgacatatgttattctggaaatattgcaaacttgaatgatcctgtgaaaatgtttggtcacccacaattgactcatgatggagtgcctatttttactaagaaaactctagaaggagatgacatgtcttgggattgtgcagcaatagggaaaaaatcaatcctaggtagatccaatagagtggagctgagcaggagacaaaaccttcaaatgaccttgatgtttcactccgttagcattgactgtatttgagcaaacagatagtgccagaacagagtcatcaatattgatgctaaaatcaatatcatcaaaaatagggatatacacatgatcgataagttgaattcccctatctctatgtgcttttttgccattagttcaacagcatgcattccagtggctctatagcttttaaatcctgatgcatcatttatcagagaatatttaagcatgagcagtatttctttgaaaccttctaaatctctagtatactgttttgctttactagtatacttagttccagcaatcctaatgctaaaggaagtgttgctatggaacattacagtgaaactcttaacaagttctatgaaatgtttggggttaaaaaacaggcaccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtgcctgttttttaaccccaaacatttcatagaacttgttaagagtttcactgtaatgttccatagcaacacttcctttagcattaggattgctggaactaagtatactagtaaagcaaaacagtatactagagatttagaaggtttcaaagaaatactgctcatgcttaaatattctctgataaatgatgcatcaggatttaaaagctatagagccactggaatgcatgctgttgaactaatggcaaaaaagcacatagagataggggaattcaacttatcgatcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccactctattggatctacctaggattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataacatatgtcatcaagatctgtccaacttgggacagcagaaaacaatacatgatgatttccaggattgtgatatgggtctgccccactataccaaaccctacagggaaacttgtggttgccctggttgatcccaacatgccatctgaaaagcaagtcattctgtcgacggcctcatgggc

SEQ ID NO: 23 - DNA, 2500 bp, Synthetic construct
  source 1..2500: mol_type = other DNA
gcccatgaggccgtcgacttattctggaaatattgcaaacttgaatgatcctgtgaaaatgtttggtcacccacaattgactcatgatggagtgcctatttttactaagaaaactctagaaggagatgacatgtcttgggattgtgcagcaatagggaaaaaatcaatcatatgaaattttaatttctcccctggcaaagtctatcttccaggaaggaatttggatgctgtctaaataagacatagtttgtgtgttagatggaagacattcaagtgtttttgaaaggaaatatttccttttgtaggcctaggtgcttcccgttgccaacaaaaagatcatttttccagtttgagatatgatggtttgtatctactatcattcttgcagaaagatcataaccctctgattctgtgatagaatcagattcataggttccaaaggaagatgatcgatacttagttccagcaatcctaatgctaaaggaagtgttgctatggaacattacagtgaaactcttaacaagttctatgaaatgtttggggttaaaaaacaggcaactagtgctctataaaataaagcatcattttcactttcatgctcatcaagtatatttctctgtctgttttggttctctgcattttctcaaaaacagaaactaggaaatctacagacccggtgacattttaccggtggccttgacggcccttctgcttttttgtacaaacttgtgatgacggtatcgataagcttgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgatcgaattcctgcagcccgggggatccactagatgcatgctcgagcggccgccagtgtgatggatatctgcagaattcgcccttatcacaagtttgtacaaaaaagcagaagggccgtcaaggccaccggtaaaatgtcaccgggtctgtagatttcctagtttctgtttttgagaaaatgcagagaaccaaaacagacagagaaatatacttgatgagcatgaaagtgaaaatgatgctttattttatagagcactagttgcctgttttttaaccccaaacatttcatagaacttgttaagagtttcactgtaatgttccatagcaacacttcctttagcattaggattgctggaactaagtatcgatcatcttcctttggaacctatgaatctgattctatcacagaatcagagggttatgatctttctgcaagaatgatagtagatacaaaccatcatatctcaaactggaaaaatgatctttttgttggcaacgggaagcacctaggcctacaaaaggaaatatttcctttcaaaaacacttgaatgtcttccatctaacacacaaactatgtcttatttagacagcatccaaattccttcctggaagatagactttgccaggggagaaattaaaatttcatatgattgattttttccctattgctgcacaatcccaagacatgtcatctccttctagagttttcttagtaaaaataggcactccatcatgagtcaattgtgggtgaccaaacattttcacaggatcattcaagtttgcaatatttccagaataagtcgacggcctcatgggc

SEQ ID NO: 24 - DNA, 23 bp, Synthetic construct
  source 1..23: mol_type = other DNA
gccaccggtcatgtgtatatccc

SEQ ID NO: 25 - DNA, 23 bp, Synthetic construct
  source 1..23: mol_type = other DNA
gccgtcgacaagttgaattcccc

SEQ ID NO: 26 - DNA, 22 bp, Synthetic construct
  source 1..22: mol_type = other DNA
gccaccggtcatcttcctttgg

SEQ ID NO: 27 - DNA, 24 bp, Synthetic construct
  source 1..24: mol_type = other DNA
gccgtcgacatacttagttccagc

SEQ ID NO: 28 - DNA, 24 bp, Synthetic construct
  source 1..24: mol_type = other DNA
ccggtatattccgacagaatccgc

SEQ ID NO: 29 - DNA, 24 bp, Synthetic construct
  source 1..24: mol_type = other DNA
ctcccctggcaaagtctatcttcc

SEQ ID NO: 30 - DNA, 20 bp, Synthetic construct
  source 1..20: mol_type = other DNA
gccaccggtcctacaaaagg

SEQ ID NO: 31 - DNA, 30 bp, Synthetic construct
  source 1..30: mol_type = other DNA
gccgtcgactgctctataaaataaagcatc

SEQ ID NO: 32 - DNA, 21 bp, Synthetic construct
  source 1..21: mol_type = other DNA
cggtgcctgttttttaacccc

SEQ ID NO: 33 - DNA, 21 bp, Synthetic construct
  source 1..21: mol_type = other DNA
gccgtcgacagaatgacttgc

SEQ ID NO: 34 - DNA, 22 bp, Synthetic construct
  source 1..22: mol_type = other DNA
cggtaaaatgtcaccgggtctg

SEQ ID NO: 35 - DNA, 23 bp, Synthetic construct
  source 1..23: mol_type = other DNA
atgaggccgtcgacttattctgg

SEQ ID NO: 36 - DNA, 20 bp, Synthetic construct
  source 1..20: mol_type = other DNA
cacgcaagtccgcatcttca

SEQ ID NO: 37 - DNA, 20 bp, Synthetic construct
  source 1..20: mol_type = other DNA
gtatcagtgtgcatggctgg

SEQ ID NO: 38 - DNA, 20 bp, Synthetic construct
  source 1..20: mol_type = other DNA
catgaagatgcggacttgcg

SEQ ID NO: 39 - DNA, 17 bp, Synthetic construct
  source 1..17: mol_type = other DNA
atccacgccgtattcgg